(19) United States
(12) Patent Application Publication
(10) Pub. No.: US 2018/0078798 A1
(43) Pub. Date: Mar. 22, 2018

(54) RESPIRATORY MASKS, SYSTEMS AND METHODS

(71) Applicant: Microsfere Pte. Ltd., Singapore (SG)

(72) Inventors: Judit FABIAN, Singapore (SG); Mark Appleton HILDESLEY, Red Beach, Auckland (NZ); William Jack MACNEISH, III, Newport Beach, CA (US); William Edwin John GRANT, St Andrews Park, Queenstown (NZ); Edwin Egge Hendrik NIEMAN, Roslyn, Dunedin (NZ); Benjamin John STRUTT, Cambridge (GB); Roberto BASILE, Hardwick, Hardwick (GB); James BAKER, West Wratting, Cambridge (GB)

(73) Assignee: Microsfere Pte. Ltd., Singapore (SG)

(21) Appl. No.: 15/564,108

(22) PCT Filed: Apr. 4, 2016
(86) PCT No.: PCT/IB2016/051898

§ 371 Date: Oct. 3, 2017

Related U.S. Application Data
(60) Provisional application No. 62/142,992, filed on Apr. 3, 2015.
(60) Provisional application No. 62/162,651, filed on May 15, 2015.

(51) Int. Cl. A62B 18/10; A62B 7/10; A62B 18/02; A62B 18/00

(52) U.S. Cl. CPC A62B 18/10; A62B 7/10; A61B 5/01; A62B 18/006; A62B 18/025

(57) ABSTRACT

A user-wearable device incorporates a respirator or breathing air filter in combination with an electronic system providing functionality to a wearing user. The functionality can include, for example, physiological data sensing, environmental data sensing, user input, user output, and communication network connectivity. The electronic system can be configured to communicate with an application executing on a user host device, such as a mobile phone, tablet or personal computer for transferring information gathered by the user-wearable device. The application executing on the user host device can be used to configure the user-wearable device. User host devices of multiple users can be configured to report gathered data to a data management system, which can aggregate and store data and perform analysis on the aggregated data. Various control arrangements may be used.

### FIELD OF THE INVENTION

The invention relates to self-contained respiratory masks, and to associated systems and methods.

### BACKGROUND TO THE INVENTION

Respirators are generally used by professionals for the single purpose of protecting themselves in specific situations, mostly in medical and industrial settings, or for consumer protection in polluted environments. Systems used in medical or industrial settings are often bulky, complex and expensive.

Respirators can be contrasted with the separate field of medical ventilators, which are used in medical settings to support the breathing of incapacitated patients. This specification is concerned solely with protective respirators.

Air pollution has become an increasing threat to humans due to industrialization and de-forestation, resulting in increasing cases of human respiratory illnesses, pollution related complications of cardiovascular conditions and conditions related to the organs, resulting in an increase in health care costs. Wearing a respiratory mask in daily life is becoming a norm in many places around the world.

Prior respiratory masks typically include an interior chamber surrounding the mouth and nose of a user, sealing against the surrounding skin. Ambient air is drawn through a filter membrane or media, which removes pollutants, into the interior chamber, and thereafter inhaled. This chamber is generally subject to changes in pressure, increased relative humidity, temperature and moist condensation during respiratory activity. Some masks operate on the principle of air entering and leaving through the same filter media. Some masks which draw intake air through filter media and evacuate it through an alternative or supplementary location such as a one-way outlet valve. Outlet valves preference a one-way flow to ensure that potentially contaminated air is not drawn into the mask chamber on the intake breath. Such outlet values typically rely on material properties and structural design to dynamically activate and seal, releasing exhaled air under positive pressure, and closing on the negative pressure of an intake breath. Typically, humidity and positive pressure in the chamber experienced by a user increases in response to the requirement to overcome the resistance of the material properties to activate the outlet valve, which results in an interrupted, less natural breathing profile, and less perceived level of general comfort when wearing the mask itself.

Self-contained respiratory masks are worn over a user's face, and generally include a mask body, harness, filters and optionally a one-way outlet valve. All components of the mask are worn on the user's head, with no external hoses or the like required for connection to external filters or fans. Some self-contained respiratory masks may be suitable for some medical and light industrial purposes, or for general use in polluted environments by workers and the general public. Self-contained respiratory masks may be worn, for example, by pedestrians, cyclists and workers in polluted cities.

Self-contained respiratory masks can be contrasted with complex, bulky and costly respirators such as prior positive pressure systems which may include a mask or helmet, external hose and bulky tanks, pumps, filters etc.

Prior self-contained respiratory masks are generally passive negative pressure masks, in which pressure created by the user's breath is used to draw fresh air into the mask and expel spent air from the mask. Prior negative pressure masks suffer from poor performance. The user is required to exert sufficient force through their breath to draw air through the filter, and to overcome the opening force of any inlet and outlet valves. Moisture contained in the user's exhaled breath tends to condense within the mask. In addition to the discomfort caused by this moisture, in some prior masks moisture deteriorates the filter performance, making it harder for the user to breath.

Some attempts have been made to improve the performance of negative pressure masks. WO2014/081788 discloses an attachable outlet fan for a negative pressure mask. The outlet fan runs continuously to draw heat and moisture out of the mask. This is said to leave fresh air rather than exhaled air in the mask as the user begins to inhale.

Reference to any prior art in this specification does not constitute an admission that such prior art forms part of the common general knowledge.

It is an object of the invention to provide improved respiratory mask user comfort and/or respiratory mask performance, or at least to provide the public with a useful choice.

### SUMMARY OF THE INVENTION

In a first aspect the invention provides a self-contained respiratory mask including: a mask body configured to be positioned over at least part of a user's face, the mask body, in use, cooperating with the user's face to define an enclosed space covering at least the user's nostrils and mouth; at least one inlet path for entry of air into the enclosed space, the inlet path including an air inlet formed in the mask body and an inlet filter; at least one outlet path for exit of air from the enclosed space, the outlet path including an outlet formed in the mask body and a controllable outlet valve; a power source; one or more sensors configured to sense one or more parameters indicative of a breathing cycle of the user; and a controller configured to control the outlet valve in accordance with the sensed parameters.

Preferably each outlet valve includes a valve member having one or more magnetic elements, a valve seat, and an electromagnet configured, when actuated, to create a force acting on the magnetic elements to drive movement of the valve member relative to the valve seat. Preferably each outlet valve includes two valve members, each having one or more magnetic elements and a valve seat, the electromagnet being arranged to drive movement of both of the two valve members.

Preferably the magnetic elements are ferromagnetic foil elements laminated to a body of the valve member. Preferably the ferromagnetic foil elements are laminated to the body of the valve member.

Preferably the body of the valve member is formed from a polymer film.

The valve member may be biased to the closed position. The valve member's bias may be provided by the structure of the valve member.

Preferably the controller is arranged to control the outlet valve, to close and hold closed the outlet valve.

In some embodiments the controller may be arranged to control the outlet valve, to release the outlet valve to allow it to open under air pressure. The outlet valve may be allowed to open under the air pressure provided by the user's breath during a normal exhalation cycle.

However, preferably the controller may be arranged to control the outlet valve, to actively open the outlet valve.

The inlet path further may include an inlet fan and the outlet valve may be arranged to open under the pressure provided by one or more of: the user's breath during a normal exhalation phase; the pressure provided by the inlet fan; and the combined pressure of the user's breath during a normal exhalation phase and the pressure provided by the inlet fan.

Preferably the controller is configured to control timed closure of the outlet valve. Preferably the controller is configured to control timed release or active opening of the outlet valve.

Preferably the inlet path further includes an inlet fan and wherein the controller is also configured to control the inlet fan in accordance with the sensed parameters.

The controller may be configured to control the inlet fan such that sufficient pressure is generated to cause airflow into the enclosed space such that pressure acting outwards from the enclosed space causes the outlet valve to open or to remain open.

Preferably the controller is configured to control one or more of: a power level of the inlet fan, and a power level applied to the closure of the outlet valve.

Preferably the controller is configured to control flow parameters of the inlet fan over the user's breathing cycle.

Preferably the controller is configured to control the outlet valve to: close the outlet valve at a desired point of the breathing cycle; and release and/or open the outlet valve at a further desired point of the breathing cycle. Preferably the controller is configured to dynamically update the desired point and/or the further desired point of the breathing cycle.

Preferably the controller is configured to control the outlet valve and/or the inlet fan to open the outlet valve at or before the beginning of the exhalation phase of the user's breathing cycle. Preferably the controller is configured to control the outlet valve and/or the inlet fan to open the outlet valve 0.01% to 12% of a breathing cycle before the beginning of the exhalation phase of the user's breathing cycle. More preferably the controller is configured to control the outlet valve and/or the inlet fan to open the outlet valve 0.01% to 5% of a breathing cycle before the beginning of the exhalation phase of the user's breathing cycle.

Preferably the controller is configured to control the outlet valve and/or the inlet fan such that the outlet valve remains open past the end of the exhalation phase of the user's breathing cycle. Preferably the controller is configured to control the outlet valve such that the outlet valve is closed before the beginning of the inhalation phase of the user's breathing cycle.

Preferably the inlet path further includes a one-way inlet valve positioned downstream of the inlet filter, the inlet valve being configured to allow flow through the inlet path into the enclosed space but not out of the enclosed space. Preferably the inlet valve is a passive valve that is caused to open and close by pressure acting upon it.

The self-contained respiratory mask may include a communications interface; wherein the controller is configured to: receive sensed parameters from the one or more sensors; and communicate the received parameters to an external device.

Preferably the controller is configured to: maintain local control data in the memory; update the local control data; receive sensed parameters from the one or more sensors; control the controllable inlet blower and/or the controllable outlet valve in accordance with the updated local control data and sensed parameters received from the one or more sensors.

The self-contained respiratory mask may include a communications interface, wherein the controller is further configured to: communicate usage data via the communications interface to an external device; receive, from the external device, update instructions based on the communicated usage data; and update the local control data in accordance with the update instructions.

In a further aspect the invention provides a self-contained respiratory mask including: a mask body configured to be positioned over at least part of a user's face, the mask body, in use, cooperating with the user's face to define an enclosed space covering at least the user's nostrils and mouth; at least one inlet path for entry of air into the enclosed space; at least one outlet path for exit of air from the enclosed space, the outlet path including an outlet formed in the mask body and a controllable outlet valve having a valve member including one or more magnetic elements, a valve seat, and an electromagnet configured, when actuated, to create a force acting on the magnetic elements to drive the valve member relative to the valve seat; a power source; one or more sensors configured to sense one or more parameters indicative of a breathing cycle of the user; a controller configured to control the outlet valve in accordance with the sensed parameters.

Preferably the electromagnet is controllable to create a force tending to close the outlet valve. The electromagnet may also be controllable to create a force tending to open the outlet valve.

Preferably each outlet valve includes two valve members, each having one or more magnetic elements and a valve seat, the electromagnet being arranged to drive movement of both of the two valve members.

Preferably the magnetic elements are ferromagnetic foil elements laminated to a body of the valve member. Preferably the ferromagnetic foil elements are laminated to the body of the valve member.

Preferably the body of the valve member is formed from a polymer film.

The valve member may be biased to the closed position.

In another aspect the invention provides a self-contained respiratory mask including: a mask body configured to be positioned over at least part of a user's face, the mask body, in use, cooperating with the user's face to define an enclosed space covering at least the user's nostrils and mouth; at least one inlet path for entry of air into the enclosed space, the inlet path including an air inlet formed in the mask body, an inlet blower and an inlet filter; at least one outlet path for exit of air from the enclosed space, the outlet path including an outlet formed in the mask body and an outlet valve; a power source; one or more sensors configured to sense one or more parameters indicative of a breathing cycle of the user; a controller configured to control the inlet fan in accordance with the sensed parameters.

Preferably the inlet path further includes a one-way inlet valve positioned downstream of the inlet filter, the inlet valve being configured to allow flow through the inlet path into the enclosed space but not out of the enclosed space. Preferably the inlet valve is a passive valve that is caused to open and close by pressure acting upon it.

In a further aspect the invention provides a self-contained respiratory mask including: a mask body configured to be positioned over at least part of a user's face, the mask body, in use, cooperating with the user's face to define an enclosed space covering at least the user's nostrils and mouth; at least one inlet path for entry of air into the enclosed space, the inlet path including an air inlet formed in the mask body, an inlet fan and an inlet filter; at least one outlet path for exit of air from the enclosed space, the outlet path including an outlet formed in the mask body and an outlet valve; a power source; one or more sensors configured to sense one or more parameters indicative of a breathing cycle of the user, the sensors including an electrical sensor configured to sense one or more electrical characteristics of the inlet fan; a controller configured to control the outlet valve and/or the inlet fan in accordance with the sensed parameters including the sensed electrical characteristics.

Preferably the controller is configured to control one or more of: timed closure of the outlet valve; timed release or active opening of the outlet valve; the inlet fan such that sufficient pressure is generated to cause airflow into the enclosed space such that pressure acting outwards from the enclosed space causes the outlet valve to open or to remain open; a power level of the inlet fan, and a power level applied to the closure of the outlet valve; flow parameters of the inlet fan over the user's breathing cycle.

Preferably the controller is configured to control the outlet valve to: close the outlet valve at a desired point of the breathing cycle; and release and/or open the outlet valve at a further desired point of the breathing cycle. Preferably the controller is configured to dynamically update the desired point and/or the further desired point of the breathing cycle.

Preferably the controller is configured to control the outlet valve and/or the inlet fan to open the outlet valve at or before the beginning of the exhalation phase of the user's breathing cycle. Preferably the controller is configured to control the outlet valve and/or the inlet fan to open the outlet valve 0.01% to 12% of a breathing cycle before the beginning of the exhalation phase of the user's breathing cycle. More preferably the controller is configured to control the outlet valve and/or the inlet fan to open the outlet valve 0.01% to 5% of a breathing cycle before the beginning of the exhalation phase of the user's breathing cycle.

Preferably the controller is configured to control the outlet valve and/or the inlet fan such that the outlet valve remains open past the end of the exhalation phase of the user's breathing cycle. Preferably the controller is configured to control the outlet valve such that the outlet valve is closed before the beginning of the inhalation phase of the user's breathing cycle.

The self-contained respiratory mask may include a communications interface; wherein the controller is configured to: receive sensed parameters from the one or more sensors; and communicate the received parameters to an external device.

Preferably the controller is configured to: maintain local control data in the memory; update the local control data; receive sensed parameters from the one or more sensors; control the controllable inlet blower and/or the controllable outlet valve in accordance with the updated local control data and sensed parameters received from the one or more sensors.

The self-contained respiratory mask may include a communications interface, wherein the controller is further configured to: communicate usage data via the communications interface to an external device; receive, from the external device, update instructions based on the communicated usage data; and update the local control data in accordance with the update instructions.

In a further aspect the invention provides a respiratory mask including a mask body configured to be positioned over at least part of a user's face, the mask body, in use, cooperating with the user's face to define an enclosed space covering at least the user's nostrils and mouth; at least one inlet path for entry of air into the enclosed space, the inlet path including an air inlet formed in the mask body, an inlet fan and an inlet filter; and at least one outlet path for exit of air from the enclosed space;

wherein the inlet fan is positioned in a fan chamber and the inlet filter is arranged to extend along at least two sides of the fan chamber for introduction of air through the inlet filter into the fan chamber, the inlet filter including a first portion extending along a first side of the fan chamber and a second portion extending at an angle to the first portion along a second wall of the fan chamber.

Preferably the filter consists of a filter material held in a filter frame that is arranged for removable attachment to the mask body.

In another aspect the invention provides a self-contained respiratory mask including: a mask body configured to be positioned over at least part of a user's face, the mask body, in use, cooperating with the user's face to define an enclosed space covering at least the user's nostrils and mouth; at least one inlet path for entry of air into the enclosed space, the inlet path including an air inlet formed in the mask body and an inlet filter; at least one outlet path for exit of air from the enclosed space, the outlet path including an outlet formed in the mask body and an outlet valve; a power source; one or more sensors configured to sense one or more parameters associated with a wearer's physiology and/or breathing cycle; a communications interface; a controller configured to: receive sensed parameters from the one or more sensors; and communicate the received parameters and/or processed data based on the received parameters to an external device.

Preferably the controller is further configured to: maintain one or more local control data in the memory; update the set of local control data; receive sensed parameters from the one or more sensors; control the controllable inlet blower and/or the controllable outlet valve in accordance with the updated local control data and sensed parameters received from the one or more sensors.

Preferably the controller is further configured to: receive, from the external device, update instructions based on the communicated parameters and/or processed data; and update the local control data in accordance with the update instructions.

In another aspect the invention provides a self-contained respiratory mask including: a mask body configured to be positioned over at least part of a user's face, the mask body, in use, cooperating with the user's face to define an enclosed space covering at least the user's nostrils and mouth; at least one inlet path for entry of air into the enclosed space, the inlet path including an air inlet formed in the mask body and an inlet filter; at least one outlet path for exit of air from the enclosed space, the outlet path including an outlet formed in the mask body; one or more of: a controllable inlet blower positioned in one of the at least one inlet paths and a controllable outlet valve positioned in one of the at least one outlet paths; a power source; one or more sensors configured to sense one or more parameters associated with a wearer's physiology and/or breathing cycle; memory; a controller configured to: maintain local control data in the memory; update the local control data; receive sensed parameters from the one or more sensors; control the controllable inlet blower and/or the controllable outlet valve in accordance with the updated local control data and sensed parameters received from the one or more sensors.

The self-contained respiratory mask may include a communications interface, wherein the controller is further configured to: communicate the received parameters and/or processed data based on the received parameters to an external device; receive, from the external device, update instructions based on the communicated parameters and/or processed data; and update the local control data in accordance with the update instructions.

The self-contained respiratory mask may include a front mask portion and a back harness portion separable at a number of connectors, at least one of the connectors providing separable mechanical and electrical connection between the front mask portion and the back harness portion.

The self-contained respiratory mask of any of the above aspects may be configured to operate in a purely passive mode in the event of power failure. Preferably, in the passive mode, the unassisted force of the user's breath draws air in through the inlet filter and forces air out through the outlet valve.

For any of the above aspects, preferably the controller is arranged to implement one of a plurality of active control modes including at least a high power mode in which mask function is prioritized and a low power mode in which power source life is prioritized.

For any of the above aspects, preferably the controller is configured to change the control mode based on input from the user. For any of the above aspects, preferably the controller is configured to change the control mode based on data received from the one or more sensors and/or information on remaining power source charge.

For any of the above aspects, preferably the one or more sensors include one or more pressure sensors. For any of the above aspects, preferably the pressure sensors include a first sensor positioned to the outside of the inlet filter and inlet fan and a second sensor positioned to the inside of the inlet filter and inlet fan. For any of the above aspects, preferably the pressure sensors include a pressure sensor in the enclosed space.

For any of the above aspects, preferably the one or more sensors include an electrical sensor configured to sense one or more electrical characteristics of the inlet fan.

For any of the above aspects, preferably the controller is configured to issue an alert when information received from the sensors indicates that the inlet filter requires cleaning or replacement.

For any of the above aspects, the respiratory mask may include a gasket arrangement on the inside of the mask body, the gasket arrangement being configured to create a seal against the user's face and to be compressed by the force applied by a harness portion.

For any of the above aspects, the respiratory mask may include one or more user input modules and/or one or more user output modules.

For any of the above aspects, preferably the user input modules comprise a microphone positioned within the enclosed space and wherein the user output modules comprise an earphone.

For any of the above aspects, the respiratory mask may include a communications interface for communications with a user's mobile device, Smartphone and/or computer.

For any of the above aspects, the respiratory mask may include one or more physiological sensors. Preferably the physiological sensors comprise one or more of: temperature sensors; a body temperature sensor; an air temperature sensor positioned to sense temperature of exhaled air; an air pressure sensor.

For any of the above aspects, the respiratory mask may include a GPS receiver module.

For any of the above aspects, the respiratory mask may include memory configured to store data gathered by at least one of the sensors.

In yet a further aspect, the invention provides a user-wearable device comprising: a respiratory mask portion configured to cover a nostrils and mouth of a user, the respiratory mask portion comprising a replaceable air filter, a device electronic system, the device electronic system comprising: a control unit, a power unit configured to provide power to the control unit, one or more user input modules, wherein at least one of the user input modules is positioned within the respiratory mask portion, one or more user output modules, and one or more communications modules, and a housing portion containing a portion of the device electronic system including at least the control unit.

Preferably the user-wearable device further comprises a frame portion to support the housing portion or the respiratory mask portion.

Preferably the frame portion and the housing portion are integral and wherein the frame portion is indirectly attached to the respiratory mask portion.

Preferably the user input modules comprise a microphone positioned within the respiratory mask portion and wherein the user output modules comprise an earphone.

Preferably the communications modules comprise a Bluetooth module.

Preferably the device electronic system further comprises one or more physiological sensors, wherein at least one of the physiological sensors is positioned within the respiratory mask portion.

Preferably the physiological sensors comprise one or more temperature sensors. Preferably the one or more temperature sensors comprise a body temperature sensor. Preferably the one or more temperature sensors comprise an air temperature sensor positioned to sense temperature of exhaled air. Preferably the physiological sensors comprise an air pressure sensor positioned within the respiratory mask portion.

Preferably the device electronic system further comprises a camera.

Preferably the user input modules comprise one or more control buttons.

Preferably the device electronic system further comprises a GPS receiver module.

Preferably the device electronic system further comprises a memory configured to store data gathered by at least one of the physiological sensors.

Preferably the device electronic system is configured to transmit the stored data to a user host device through one or more of the communications modules.

Preferably the control unit comprises a CPU and memory. Preferably the control unit comprises a microcontroller.

### BRIEF DESCRIPTION OF THE DRAWINGS

The invention will now be described by way of example only, with reference to the accompanying drawings, in which:

FIG. 1 is a perspective side view of a user-wearable device being worn by a person in accordance with one embodiment.

FIG. 2 is a perspective side view of the user-wearable device in accordance with one embodiment.

FIG. 3 is a perspective front view of the user-wearable device in accordance with one embodiment.

FIG. 4 is a functional block diagram of a device electronic system in accordance with one embodiment.

FIG. 5 is a system diagram showing multiple user-wearable devices of multiple users in communication with user host devices, which are in turn in communication with a data management system in accordance with one embodiment.

FIG. 6 is a block diagram of a general purpose computer in accordance with one embodiment.

FIG. 7 is a side view of a further embodiment of respiratory mask.

FIG. 7A is a front view of the mask of FIG. 7.

FIG. 7B is a bottom view of the mask of FIG. 7.

FIG. 7C is a top view of the mask of FIG. 7.

FIG. 7D is a back view of the mask of FIG. 7.

FIG. 7E is a side view of the mask of FIG. 7, in a partly disassembled state.

FIG. 7F is a perspective rear view of the mask of FIG. 7.

FIG. 7G is a front view of the mask of FIG. 7, showing the mask worn by a user.

FIG. 7H is a side view of the mask of FIG. 7, showing the mask worn by a user.

FIG. 7I is a back view of the mask of FIG. 7, showing the mask worn by a user.

FIG. 7G is a side view of the mask of FIG. 7, showing a back frame or harness portion of the mask worn by a user independent of the front mask portion.

FIG. 8 is a perspective view of an outlet valve according to one embodiment.

FIG. 8A is a further perspective view of the valve of FIG. 8, showing the valve members in an open position.

FIG. 8B is a side view of the valve of FIG. 8.

FIG. 8C is a cross-section along the line B-B in FIG. 6B.

FIG. 8D is a side view of the valve of FIG. 8.

FIG. 8E is a cross-section along the line C-C in FIG. 8D.

FIG. 8F is an end view of the valve of FIG. 8.

FIG. 8G is a cross-sectioon along the line D-D inb FIG. 8F.

FIG. 9 is a functional schematic diagram of a respiratory mask according to one embodiment.

FIG. 10 shows an inlet filtegr and fan according to one embodiment.

FIG. 11 is a simplified diagram illustrating a user's breathinbg cycle and timing of certain mask functions during the breathing cycle.

FIG. 12 is a front view of a mask portion, showing an inlet valve in an open position.

FIG. 12A is a further front view of the mask portion of FIG. 12, showing the inlet valve in a closed position.

FIG. 12B shows the inlet valve of FIG. 12, in an open position.

FIG. 12C shows the inlet valve of FIG. 12, in a closed position.

### DETAILED DESCRIPTION

In the following description, reference is made to the accompanying drawings, which form a part hereof, and which show, by way of illustration, specific embodiments or processes in which the invention may be practiced. Where possible, the same reference numbers are used throughout the drawings to refer to the same or like components. In some instances, numerous specific details are set forth in order to provide a thorough understanding of the present invention. The present invention, however, may be practiced without the specific details or with certain alternative equivalent devices, components, and methods to those described herein. In other instances, well-known devices, components, and methods have not been described in detail so as not to unnecessarily obscure aspects of the present invention.

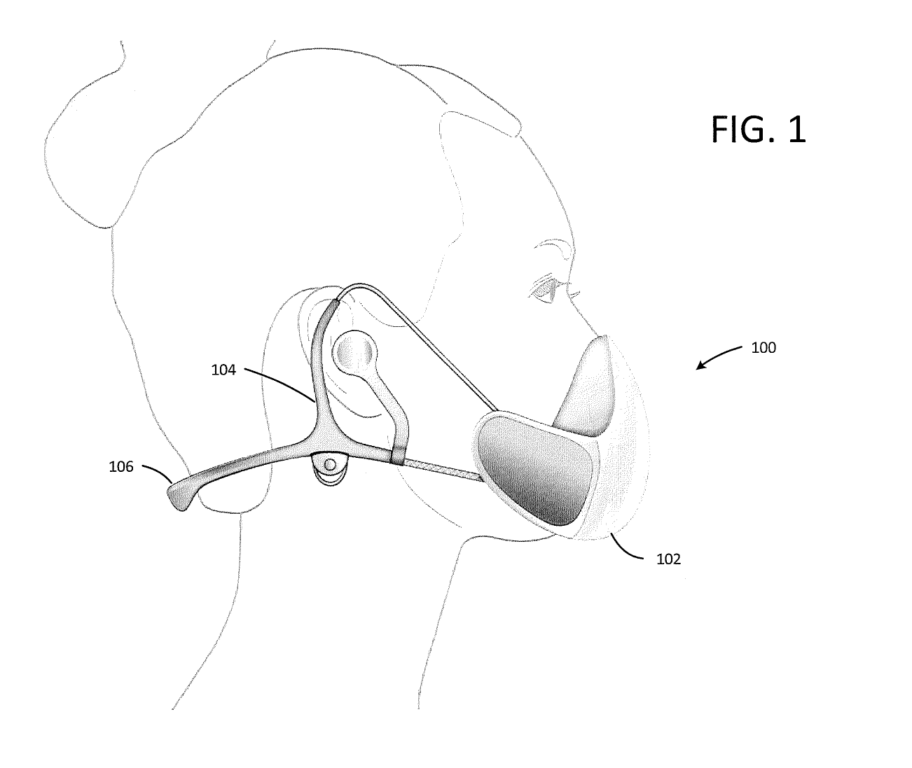

FIG. 1 is a perspective side view of a user-wearable self-contained respiratory mask device 100 being worn by a person in accordance with one embodiment. The user-wearable respiratory mask device 100 includes a mask portion 102 configured to cover the nostrils and mouth of the user and to provide breathing air filtration functionality. The device 100 can include a frame portion 104 configured to provide support for holding the respiratory mask portion 102 against the user's face. The frame 104 may support the device 100 on the user's head using an appropriate harness, straps or the like, positioned appropriately around the user's ears and/or the top and/or back of the user's head. The harness may include any suitable combination of rigid, semi-rigid and/or flexible components.

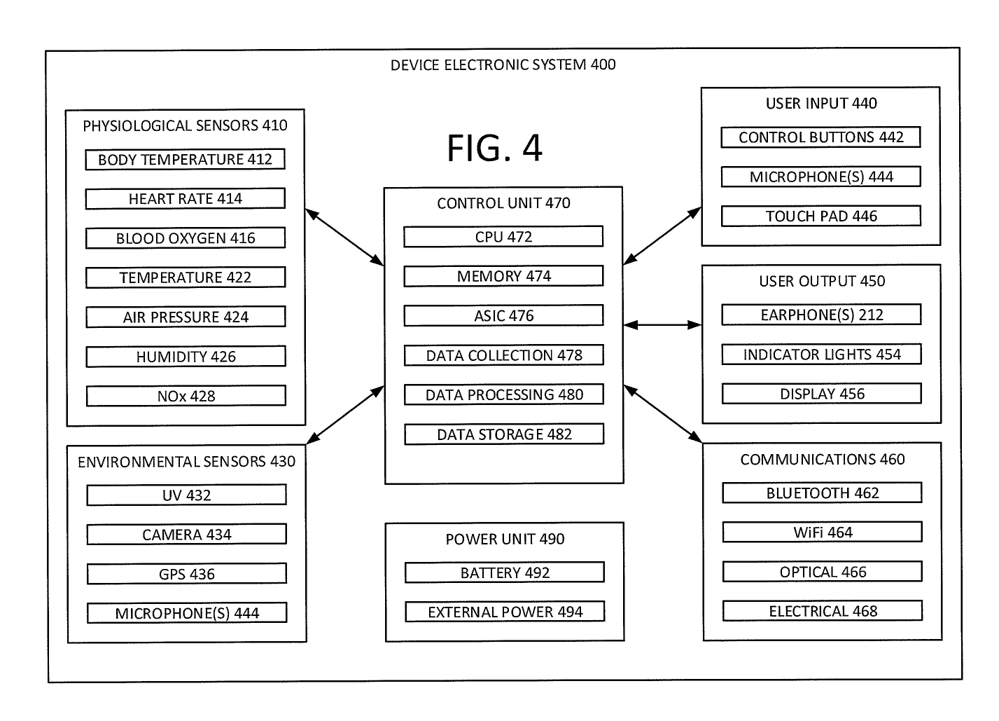

The device 100 can also include a housing portion 106 configured to house components of a device electronic system 400 (FIG. 4). In one embodiment, the frame portion 104 and the housing portion 106 can be integrated as a single unit with componentry of the device electronic system 400 inserted in or housed on various internal or external portions of the frame portion 106. In one embodiment, the frame portion 104 can be omitted or supplemented, using alternative means for securing the mask portion 102 to the user's face. In one embodiment, the housing portion 106 can be separate from and optionally secured to the frame portion 104 at any convenient location. The frame portion 104 can be configured to support the housing portion 106.

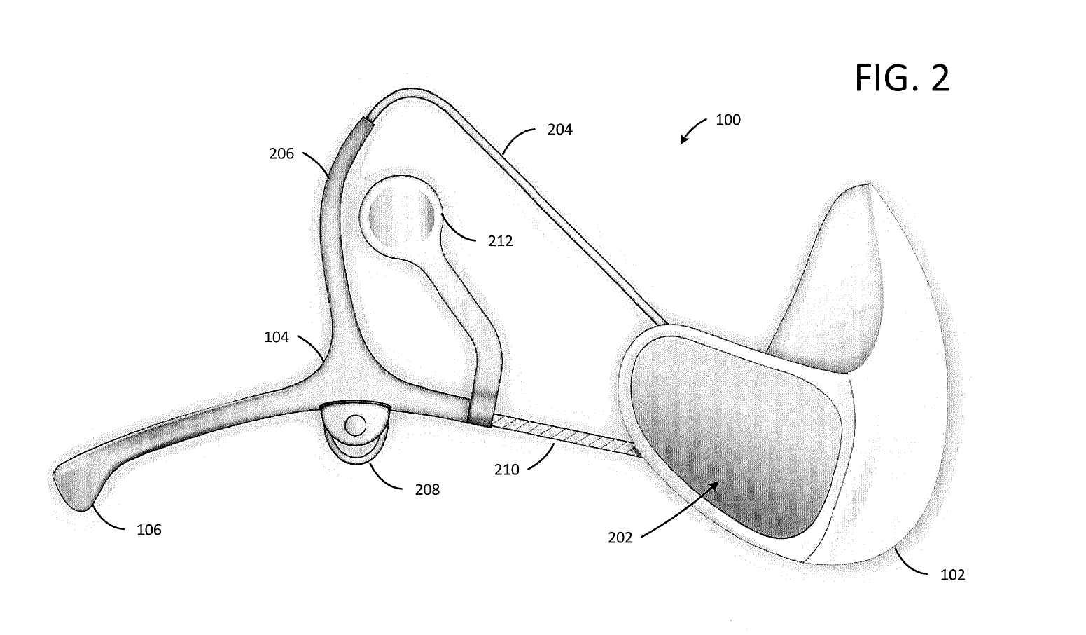

FIG. 2 is a perspective side view of the user-wearable device 100 in accordance with one embodiment. The mask portion 102 can include a replaceable air filter 202. The filter 202 can be placed on one side of the respiratory mask portion, one filter 202 can be placed on each of two sides, or the whole mask portion can be made of or covered with filter material with an optional exhalation valve.

In accordance with one embodiment, part or substantially all of the mask portion 102 can be constructed of a transparent and optionally rigid or semi-rigid material, such as silicone or hard plastic, so that the user's nose and/or mouth can be seen through the mask portion. In accordance with one embodiment, the mask portion 102 incorporates or houses physiological sensors 410 (FIG. 4) for monitoring physiological conditions of the user. The mask portion 102 can also include a microphone 444 (FIG. 4) for recording and/or transmitting the user's voice. The physiological sensors 410 and microphone 444 can be connected to the device electronic system 400 by wires, wireless or flexible printed circuits.

In accordance with one embodiment, a top of the mask portion 102 can be adjustably secured to the frame portion 104 by two cords 204, one on each of a left and right sides of the device 100. The cords 204 can be made of an elastic or inelastic material and in one embodiment, the cords include a polymer material. Each of the cords 204 can extend from the top of the mask portion 102, to an upwardly extending arm 206 of the frame portion 104. The upwardly extending arm 206 can fit behind the user's ear while the cord 204 can wrap over the top of the ear and then down to the mask portion 102. In one embodiment, each cord 204 can run down through the upwardly extending arm 206, which can be hollow, and down to an adjustment device 208. The adjustment device 208 can maintain tension on the cord 206, but also allow the cord to be adjusted in or out by user manipulation. In one embodiment, the cords 204 can be fixed to the top of the arms 206 and adjustably connected to the mask portion 102.

In one embodiment a bottom of the mask portion 102 can be secured to the frame portion 104 by an extension arm 210, one on each of the left and right sides of the device 100. The extension arm 210 can be hollow and configured to house wires or a flexible printed circuit board for connecting the sensors 410 to the device electronic system 400. The extension arm 210 can be flexible, rigid, or elastic in accordance with a desired design and fit of the wearable device 100.

In one embodiment, the user device 100 includes an earphone 212 for each or both of the user's ears. The earphone 212 can be connected to or can be part of the device electronic system 400 and can provide audio for music, phone conversations or interactive voice response features. In accordance with one embodiment, the earphone 212 can be configured to be rotated out of the ear toward the neck in case the user does not wish to use the earphone.

The mask portion 102, the frame portion 104, or the housing portion 106 can also be configured to include or house various environmental sensors 430 (FIG. 4) at any convenient locations on the user device 100. The environmental sensors 430 can be connected to the device electronic system 400 by wires, wireless or flexible printed circuits. The frame portion 104 can be configured to house various other components of the device electronic system 400 (FIG. 4) such as a battery or power unit 490 (FIG. 4), one or more communications modules 460 (FIG. 4), and a control unit 470 (FIG. 4) at any convenient locations.

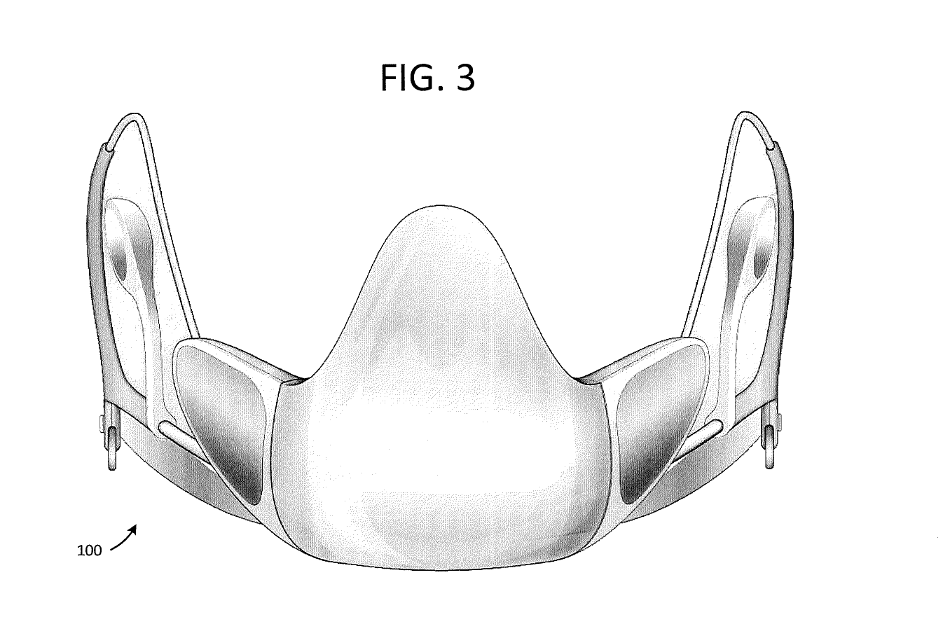

FIG. 3 is a perspective front view of the user-wearable device 100 in accordance with one embodiment.

FIG. 4 is a functional block diagram of a device electronic system 400 in accordance with one embodiment.

The device electronic system 400 can include one or more physiological sensors 410 configured to monitor physiological properties of the body of a user of the device 100. The physiological sensors 410 can include, for example, a skin or body temperature sensor 412, a heart rate sensor or monitor 414, and a blood oxygen sensor or pulse oximeter 416. The heart rate sensor may be an optical sensor arranged to sit over the wearer's mastoid process. The physiological sensors 410 can also include, for example, a temperature sensor 422, an air pressure sensor 424 (preferred embodiments may include several pressure sensors, as discussed below), a humidity sensor 426, an accelerometer, a carbon monoxide and/or carbon dioxide sensor and a nitrogen oxide (NOx) sensor 428.

In one embodiment a microphone can be placed behind the ear to detect noises indicative of physiological functions, such as breathing, coughing, heart rate, etc.

The location of the heart rate sensor in the preferred embodiment may be on either or both sides of the skull behind the ear on the Mastoid Process. The heart rate sensor will be held off a slight distance from the skin (1 mm) in this instance. The heart rate sensor will acquire the heart rate information using optical methods in the preferred embodiment, but other methods do exist.

The skin temperature sensor may be located at the same approximate location as the heart rate sensor, or may be at the same point on the other side of the skull. The hardware feature where these two types of sensors could be installed are big enough to accommodate both sensors on either side of the skull, so one sensor can be located on each side, or both could be located on either side of the skull. The receiver/holder of the heart rate and temperature sensor components is built into the back side connector edges of the mask back part. The micro controller and removable battery are built into the back edge of the back frame in the preferred embodiment. The wiring to connect to the front of the mask and the side sensors are built into the frame of the mask connector and connected between the front and back frame by connector elements.

In one embodiment, some or all of the physiological sensors can be placed in or on the respiratory mask portion 102 to collect information from, on or near the user's nose and/or mouth. For example, the air pressure sensor 424 can be positioned within the respiratory mask portion 102, and pressure readings can be analyzed, for example, to determine a user's breathing rate or otherwise characterize the user's breathing. The temperature sensor 422 can be positioned to measure the temperature of air being inhaled or exhaled. The NOx sensor can be arranged to detect the presence of inflammation in the respiratory system. In accordance with one embodiment, body temperature can be estimated or determined based on measured temperature of exhaled air. In accordance with one embodiment, some of the physiological sensors 410 can be placed on the frame to collect the signals from behind the ear. For example, the heart rate monitor 414 and the blood oxygen detector 416 can be positioned to take readings from behind the user's ear.

The device electronic system 400 can include one or more environmental sensors 430 to monitor environmental conditions around the user. The environmental sensors 430 can include, for example, an ultraviolet light (or other radiation) sensor 432. The environmental sensors 430 can also include a camera 434, which can be configured to capture a user's perspective view of the environment. The environmental sensors 430 can also include a GPS signal receiver and/or processor module 436, although the GPS signal receiver/module can also be considered a communications module. The environmental sensors 430 can also include a magnetometer, an accelerometer and a gyroscope. The environmental sensors 430 can be located in or on the respiratory mask portion 102, the frame portion 104, the housing portion 106, or in any convenient location on the device 100.

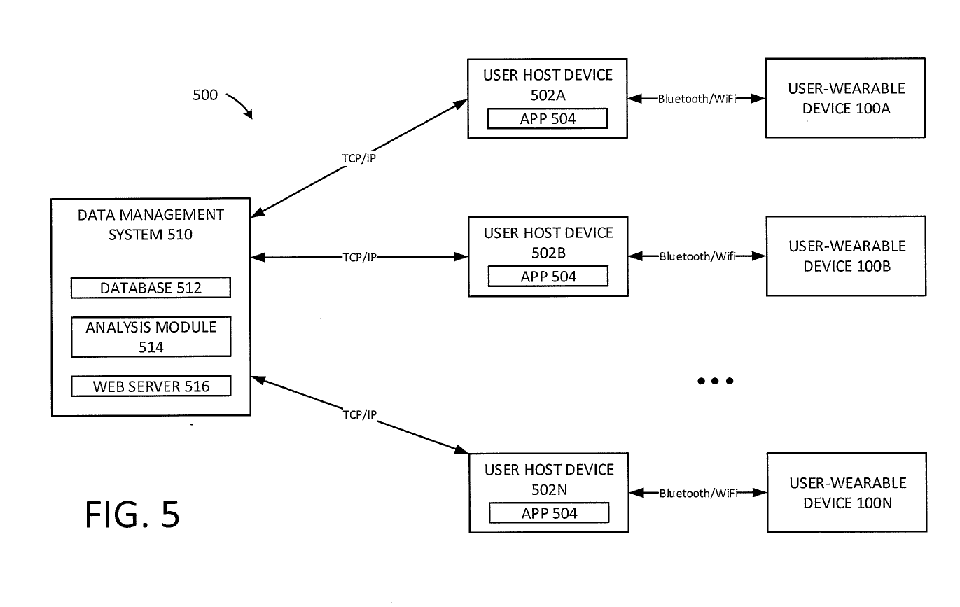

The device electronics system 400 can include one or more user input modules 440 through which a user can provide input to the device electronic system 400. The user input modules 440 can include one or more control buttons 442. The control buttons 442 can be located in or on any practical location on the device 100, such as on the frame portion 104. The user input modules 440 can include one or more microphones 444. The microphone(s) 444 can be located in or on any practical location on the device 100. In one embodiment, at least one microphone is located within the respiratory mask portion 102 to directly capture a user's spoken voice. The microphone(s) 444 can also be used to capture additional sounds produced by the user, such as from breathing, laughing or coughing, where a coughing pattern can be used for medical diagnosis or analysis. The microphone(s) 444 can also be used to provide voice control of functionality either for the mask or for another connected device, such as a user host device 502 (FIG. 5). Additional microphones 444 can be located outside the respiratory mask portion 102 to capture environmental sounds. The user input modules 440 can include a cognitive sensor or sensors, arranged to sense a user's cognitive patterns as an unspoken signal for selection or input. The user input modules 440 can include a touch pad 446, such as touch sensitive a trackpad or dial pad. The touch pad 446 can be located in or on any practical location on the device 100.

The device electronics system 400 can include one or more user output modules 450 through which the device electronic system 400 can output information to a user of the device or to other people in proximity to the user. The user output modules 450 can include one or two earphones 212. The earphones 212 can be integral with the device 100, as illustrated in FIGS. 1-3, or the earphones 212 can be user-supplied and plugged into an earphone jack on the device 100. The user output modules 450 can include one or more indicator lights 454. The indicator lights 454 can be located in or on any practical location on the device 100. The indicator lights 454 can be used to indicate various operating conditions of the device such as, for example, power on status, battery status, or in phone call status. The user output modules 450 can include a display 456. The display 456 can be located in or on any practical location on the device 100. In one embodiment, the display 456 can be an LED or LCD display for displaying various operating conditions of the device. In one embodiment, the display 456 can be configured to display information or aesthetic visuals to people other than the person wearing the device 100. The display can be alphanumeric, graphic and/or semiconductor color display, such as LED, elnk display. The display 456 can range from a simple indicator light to a full screen display with alphanumeric, graphic and video capabilities. The user output module can include a speaker that the user can utilize to transmit his/her voice or play other sounds (music, recorded speech, recorded noises, etc.)

The device electronics system 400 can include one or more communications modules 460 through which the device electronic system 400 can effect communications with other devices or through a communications network. The communications modules 460 can include one or more of: a Bluetooth module 462, a WiFi module 464, an optical (e.g. wireless infrared or fiber optic) transceiver module 466, and an electrical communications module 468. The electrical communications module 468 can be, for example, an Ethernet network interface. A USB port can be included to provide communications connectivity to other devices and/or to provide battery charging functionality. Additional communications modules 460 can include, for example, NFC, Zigbee or other short or long range wireless transmission technologies. The communications modules 460 can also include a GSM module for direct connection to the mobile network.

The Bluetooth module 462 can be integrated with or connected to the microphone 444 and earphones 212 to provide mobile phone call functionality and/or audio playback functionality. Additional features supported through a Bluetooth connection can also be provided, such as Bluetooth data synchronization or data transfer, Bluetooth mouse functionality through the touchpad 446 or Bluetooth voice control of a connected device, such as a user host device 502 (FIG. 5).

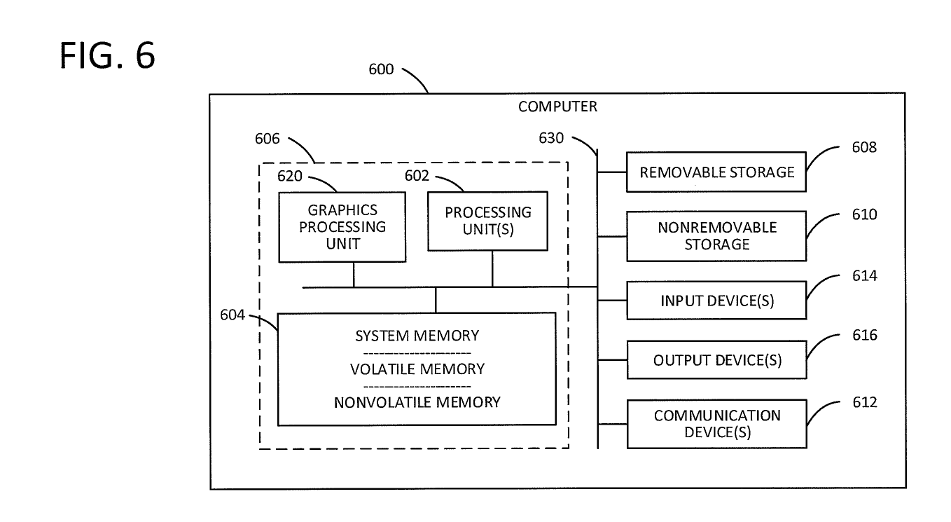

The device electronics system 400 can include a control unit 470 connected to and configured to control and operate the various sensors, user input modules and user output modules. The control unit 470 can include a CPU 472 and memory 474 or a microcontroller. The CPU 470 and memory 472 can be configured to execute an operating system and applications to provide control of an access to the device's features. In one embodiment the control unit 470 can include one or more application specific integrated circuits (ASIC) 476 configured to provide the control and access functionality in addition to or instead of the CPU 472 and memory 474. In accordance with one embodiment, the control unit 470 can be implemented wholly or in part using a general purpose computer 600 as discussed below with reference to FIG. 6.

In one embodiment, the control unit 470 can include a data collection module 478, a data processing module 480 and a data storage module 482. In one embodiment, the data collection module 478, the data processing module 480 and the data storage module 482 can be implemented through a combination of one or more of the CPU 472, the memory 474, and/or the ASICs 476. The data collection module 478 can be configured to receive signals from the sensors and user input modules in digital and/or analog format. The data processing module 480 can be configured to process the received signals to produce data in a format that can be stored and transmitted. The data storage module 482 can be configured to store processed or unprocessed data for subsequent processing, use, or transmission. In one embodiment, the data storage module 482 is implemented using the memory 474.

The device electronics system 400 can include a power unit 490 configured to provide power to the control unit 470, the sensors, the user input and output modules and the communications modules. The power unit 490 can include a battery 492, an external power supply port 494, or both. The external power supply port 494, which can be a USB port, can be used to charge the battery 492. The battery 492 and the external power supply port 494 can be located in or on any practical location on the device 100, such as on the frame portion 104 or in the housing portion 106. The power unit 490 can optionally include one or more solar cells or an induction charging module for charging the battery 492 and/or for providing ongoing power.

In accordance with one embodiment, the control unit 470 can be configured to collect, process, and transmit data using the communications modules 460 so as to use power efficiently. The frequency and duration of sampling from each sensor and the frequency of upload of data to a user host device 502 (FIG. 5) can be set to optimize data collection and transmission. Different processes can be implemented by the device to collect data at different rates depending on user requirements.

FIG. 5 shows a system 500 in accordance with one embodiment where multiple user-wearable devices 100A-N of multiple users communicate with associated user host devices 502A-N, which in turn communicate with a data management system 510. Each user host device 502 can be, for example, a smartphone, a tablet computer, or a personal computer. In accordance with one embodiment, the user host device 502 and the data management system 510 can each be implemented wholly or in part using a general purpose computer 600 as discussed below with reference to FIG. 6.

Each user of a user-wearable device 100 can connect a respective wearable device 100 in communication with an associated user host device 502. The connection can be a wireless connection, such as Bluetooth or WiFi. The host device 502 can operate an application or app 504 to interface with the user-wearable device 100 in order to retrieve data collected by the user-wearable device 100. The data can be stored by the user-wearable device and forwarded to or retrieved from the host device 502 periodically or upon connection. The data can be streamed by the user-wearable device in real time. The app 504 can provide user access to data measured by the various sensors of the user-wearable device. The access can be provided to the user by displaying the data to the user or by providing the capability to export or transmit the data to destinations external to the app 504 or to the host device 502.

In one embodiment, the app 504 can be used to configure various features of the user-wearable device 100 using an application program interface through the wireless connection.

Configurations properties of the device 100 can include date/time setup, sensors setup, data sampling duration or frequency, display content, user information, and frequency of upload to the host device 502. Further, in some embodiments control data may be transmitted from the host device to the respiratory mask device, as discussed below.

In one embodiment, one or more user host devices 502 of one or more users are configured to transmit data gathered by the associated user-wearable devices 100 to a data management system 510. The communication between the host devices 502 and the data management system 510 can be through TCP/IP or other networking protocols, optionally implemented through any TCP/IP communications infrastructure, including mobile wireless. The data management system 510 can be configured to store and log data provided by multiple user-wearable devices 100, possibly in association with location data (e.g. provided by the GPS module 436 or by the host device 502) in a database 512. The data in the database 512 can be analyzed by an analysis module 514 in order to identify trends in environmental or physiological data affecting populations of people. When the body temperatures of large numbers of people in a certain geographic region are identified as being high, this might be interpreted by the analysis module 514 as an outbreak of some contagious disease or an epidemic, for example. The data management system 510 can include a web server 516 that can make data from the database 512 and analysis results from the analysis module 514 available to users through World Wide Web access.

In one embodiment the user host device 502 can gather additional data, such as GPS or geolocation data, accelerometer data, gyroscopic or magnetometer data using sensors or receivers incorporated in the user host device. This additional data gathered by host device 502 can also be combined with data from the user-wearable device 100 to provide extended functionality or a richer set of information to the user about their performance, health status or their environment. In addition, such additional data gathered by host device 502 can also be transmitted to the data management system 510 in addition to, in combination with, or to supplement data from the associated user-wearable devices 100. By gathering this data on the host device 502 and/or transmitting this additional data to the data management system 510, some sensors or receivers, such as GPS, if included in the host device 502, can be omitted from the user device 100.

In one embodiment, the app 504, residing on the user host device 502, can provide various functionality to the user. The app 504 can be configured to collect data from various sensors from the user-wearable device 100 and interpret and exhibit information to the user in an easily understandable format. Such information can include information regarding the user's athletic performance (including heart rate, breathing rate, pace, distance etc), indications of health status wellness or lifestyle related information, or feedback regarding the use of the user-wearable device 100. For example, the user can be informed of the long term improvement of their athletic performance. The app 504 can also be configured to provide information received from the data management system 510 either in a standalone manner or in combination with data gathered from the user-wearable device 100 and/or the user host device 502.

FIG. 6 is a block diagram of a general purpose computer with computer programs providing instructions to be executed by a processor in the general purpose computer. Computer programs on a general purpose computer generally include an operating system and applications. The operating system is a computer program running on the computer that manages access to various resources of the computer by the applications and the operating system. The various resources generally include memory, storage, communication interfaces, input devices and output devices.

Examples of such general purpose computers include, but are not limited to, larger computer systems such as server computers, database computers, desktop computers, laptop and notebook computers, as well as mobile or handheld computing devices, such as a tablet computer, hand held computer, smart phone, media player, personal data assistant, audio and/or video recorder, or wearable computing device.

With reference to FIG. 6, an example computer 600 includes at least one processing unit 602 and memory 604. The computer can have multiple processing units 602 and multiple devices implementing the memory 604. A processing unit 602 can include one or more processing cores (not shown) that operate independently of each other. Additional co-processing units, such as graphics processing unit 620, also can be present in the computer. The memory 604 may include volatile devices (such as dynamic random access memory (DRAM) or other random access memory device), and non-volatile devices (such as a read-only memory, flash memory, and the like) or some combination of the two. This configuration of memory is illustrated in FIG. 6 by dashed line 606. The computer 600 may include additional storage (removable and/or non-removable) including, but not limited to, magnetically-recorded or optically-recorded disks or tape. Such additional storage is illustrated in FIG. 6 by removable storage 608 and non-removable storage 610. The various components in FIG. 6 are generally interconnected by an interconnection mechanism, such as one or more buses 630.

A computer storage medium is any medium in which data can be stored in and retrieved from addressable physical storage locations by the computer. Computer storage media includes volatile and nonvolatile memory devices, and removable and non-removable storage media. Memory 604 and 606, removable storage 608 and non-removable storage 610 are all examples of computer storage media. Some examples of computer storage media are RAM, ROM, EEPROM, flash memory or other memory technology, CD-ROM, digital versatile disks (DVD) or other optically or magneto-optically recorded storage device, magnetic cassettes, magnetic tape, magnetic disk storage or other magnetic storage devices. Computer storage media and communication media are mutually exclusive categories of media.

The computer 600 may also include communication device(s) 612 through which the computer communicates with other devices over a communication medium such as a computer network. Communication media typically transmit computer program instructions, data structures, program modules or other data over a wired or wireless substance by propagating a modulated data signal such as a carrier wave or other transport mechanism over the substance. The term “modulated data signal” means a signal that has one or more of its characteristics set or changed in such a manner as to encode information in the signal, thereby changing the configuration or state of the receiving device of the signal. By way of example, and not limitation, communication media includes wired media such as a wired network or direct-wired connection, and wireless media include any non-wired communication media that allows propagation of signals, such as acoustic, electromagnetic, electrical, optical, infrared, radio frequency and other signals.

Communications device(s) 612 can include, for example, a network interface or radio transmitter, that interface with the communication media to transmit data over and receive data from signals propagated through communication media. The communication device(s) 612 can include one or more radio transmitters for telephonic communications over cellular telephone networks, and/or wireless connections to a computer network. For example, a cellular connection, a WiFi connection, a Bluetooth connection, and other connections may be present in the computer. Such connections support communication with other devices, such as to support voice or data communications.

The computer 600 may have various input device(s) 614 such as a various pointer (whether single pointer or multipointer) devices, such as a mouse, tablet and pen, touchpad and other touch-based input devices, image input devices, such as still and motion cameras, audio input devices, such as a microphone, and various sensors, such as accelerometers, thermometers and the like, and so on. Output device(s) 616 such as a display, speakers, printers, and so on, also may be included. All of these devices are well known in the art and need not be discussed at length here.

The various storage 610, communication device(s) 612, output devices 616 and input devices 614 can be integrated within a housing of the computer, or can be connected through various input/output interface devices on the computer, in which case the reference numbers 610, 612, 614 and 616 can indicate either the interface for connection to a device or the device itself as the case may be.

An operating system of the computer typically includes computer programs, commonly called drivers, that manage access to the various storage 610, communication device(s) 612, output devices 616 and input devices 614. Such access generally includes managing inputs from and outputs to these devices. In the case of communication device(s), the operating system also may include one or more computer programs for implementing communication protocols used to communicate information between computers and devices through the communication device(s) 612.

Any of the foregoing aspects may be embodied in one or more instances as a computer system, as a process performed by such a computer system, as any individual component of such a computer system, or as an article of manufacture including computer storage in which computer program instructions are stored and which, when processed by one or more computers, configure the one or more computers to provide such a computer system or any individual component of such a computer system. A server, computer server, a host or a client device can each be embodied as a computer or a computer system. A system or computer system can include multiple computers or multiple computer systems connected by a computer network.

Each component (which also may be called a “module” or “engine” or the like), of a computer system such as described herein, and which operates on one or more computers, can be implemented using the one or more processing units of the computer and one or more computer programs processed by the one or more processing units. A computer program includes computer-executable instructions and/or computer-interpreted instructions, such as program modules, which instructions are processed by one or more processing units in the computer. Generally, such instructions define routines, programs, objects, components, data structures, and so on, that, when processed by a processing unit, instruct the processing unit to perform operations on data or configure the processor or computer to implement various components or data structures.

In some embodiments, one or more actively controlled power-assisted ventilation valves are added to a respirator to improve the wearing user's experience. An inlet valve can be located between the respirator's filter media and an inside chamber to isolate the filter media from a user's exhaled breath. An outlet or exhalation valve can be located between the inside chamber and the environment to vent the user's exhaled breath. The valves can include a one-way valve in combination with a fan to effect or assist the movement of air through the valve and the respirator. The one-way valve and the fan can be actively controlled by a microcontroller based on sensor readings such as pressure, temperature or humidity, or any combination of those sensor readings. The microcontroller can be configured to preemptively and periodically activate the valve and/or fan in advance of any instance of a sensor-detected change to account for a user's monitored cyclical breathing pattern. In preferred embodiments a single fan may be arranged to exert pressure on both the inlet valve and outlet valve. A single fan may be positioned in the inlet path and exert pressure on the outlet valve, as will be discussed below.

Respirators used for filtering breathing air for human use can suffer from breathability issues. Breathability is a combination of scientific and subjective perceptual factors. These technical and perceptual contributing factors of breathability can be improved to achieve an aggregated positive change in user perceptions.

Moving air requires energy. The amount of energy required to move a certain volume of air is proportional to the volume of air multiplied by the pressure drop. Humans do not notice the pressure drop associated with breathing through the cross section of the trachea. An increased pressure drop when wearing a respirator, however, decreases breathability. Humidity can increase perceptions of claustrophobia and stuffiness, as well as raise temperature of and condensation on the skin locally, decreasing breathability. Increased temperature of materials can increase discomfort, decreasing breathability. Keeping filter media dry and clean can reduce odor and pressure drop, increasing breathability.

In one embodiment, an actively actuated non-return inlet valve is incorporated between the filter media and an inside chamber. In one embodiment an actively actuated exhaust valve passes exhaled breath to the environment. Pressure, temperature or humidity sensors, or any combination of those sensors, provide readings to a microcontroller, which operates the valve or predictively determines when to operate the valves based on a user's breathing pattern. The inlet valve or the exhaust valve can be actively or passively actuated (e.g. an umbrella or flap valve actuated by pressure).

The inlet valve and/or the exhaust valve can be supplemented by a fan to blow or suck filtered air into the chamber. The use of an inlet fan reduces or eliminates the work that needs to be done by the user to pull air through the inlet filter. Further, air introduced by the inlet fan may provide cooling and comfort, or may agitate the enclosed volume of air in such a way it gives the impression of cooling. The fan can be controlled by a microcontroller and operable in different modes and speeds either directly, through the microcontroller, or using another device connected to the microcontroller, such as a smartphone, for example, to manage operation of the fan. The fan can be configured, for example, to run full time, in an automated mode balancing comfort and power use, in a battery saver mode, in sports mode, or in a summer mode to account for high heat and/or humidity. In preferred embodiments, multiple modes of operation are provided, each involving particular control of the fan(s) and valve(s). Multiple fans can also be used and each fan can be positioned before or after the inlet valve, the exhaust valve or the filter media.

Fans assisting inhaled or exhaled air can overcome the pressure drop of a filter. Control systems implemented in the microcontroller can be used to control the fan(s) to eliminate constant positive pressure and increase comfort. The active valves can be configured to respond to natural breathing patterns as a way of assisting rapid exit of warm, humid air and can help overcome the material limitations (and resulting pressure drop) of inactive valve systems.

In accordance with one embodiment, an active valve device is incorporated into an outlet air conveying system of a respirator. The active valve can be electrically operated and utilizes sensors that combined together with a suitable electronic system can be useful in reducing the pressure drop and other negative side effects perceived by a user during the exhale phase when wearing a mask in human breathing. The active valve device can further reduce the humidity and temperature inside the respirator quickly evacuating the exhaled air, and further overcoming the pressure drop provided by a non-active system, thus improving the overall perceived comfort in wearing the respirator. The active valve can work in isolation or in combination with an active micro fan to further improve performance.

In one embodiment the air may be directly drawn through a filtration media by a fan from an external environment into the enclosed chamber in addition to any of the aforementioned combinations.

Referring to FIG. 1, an active valve device 100 includes a set of sensors 102 configured to measure relevant indicators such as pressure, flow, humidity or temperature or any combination of those indicators. Other additional sensors, which further assess key metrics to aid performance of the valve singly or in combination, can be included. The active valve device 100 can include an air flow conveying system 104, such as a frustoconical aperture, that uses features to assist directional airflow. The device 100 can also include a micro fan 106 and an electrically operated valve 108.

An electronic system 110 gathers data from the sensors 102 can be configured to algorithmically respond to sensed changes in key metrics including for example pressure, flow, temperature and humidity typically associated with exhaled air to operate the valve 108, opening to help evacuate the air flowing from inside the mask chamber (direction 112). The valve can be operated by a range of technologies including, for example, a servo motor or other electromechanical actuator.

The electronic system 110 can be configured to respond to sensed changes in for example pressure, flow, temperature or humidity (or any combination of those indicators) associated with pressure neutral, transition to or beginning of inhaled air, thus effectively blocking contaminating air from entering the mask interior while filtered air is drawn from other locations in the mask assembly.

In one embodiment, the electronic system 110 operates an additional micro fan 106 which works continuously or in combination with the valve 108 to help evacuate the air coming from direction 112 (chamber or inner part of the respirator). The micro fan 106 has additional advantages of providing a cooling sensation to the skin, whether or not used as an aid to exiting air, reducing humidity or other aforementioned advantages. For the purposes of this invention it is envisaged that the key features described may be used separately or in combination.

In accordance with one embodiment, the respirator is augmented with one or more actively controlled power-assisted ventilation valves. One valve can be located between the respirator's filter media and an inside chamber to isolate the filter media from a user's exhaled breath. Another valve can be located between the inside chamber and the environment to vent the user's exhaled breath. The valves can include a one-way valve in combination with a fan to effect or assist the movement of air through the valve and the respirator. The one-way valve and the fan can be actively controlled based on sensor readings such as temperature or humidity (or any combination of those sensor readings) by a microcontroller, which can be incorporated into the device electronics system 400. The microcontroller can be configured to preemptively and periodically activate the valve and/or fan in advance of any instance of a sensor-detected change to account for a user's monitored cyclical breathing pattern.

Existing masks are generally either passive or fully powered. In passive masks the airflow is solely dependent on the user's breathing. The simplest passive masks are simply made of a filter material with the user breathing in and out through the filter material. Slightly more sophisticated masks may have a passive outlet valve, where the valve is opened by the pressure of the user's breath acting against a valve member, and closed by a spring force acting towards a closed position. However, such prior masks are plagued by numerous comfort problems.

Prior passive masks use flap valves that open with the force of the exhalation and close as soon as the pressure from exhalation subsides to a level that cannot maintain the valve's open position. This point will generally occur towards the end of exhalation but before exhalation is fully complete. Each passive flap valve has to have enough resistive force built into the mechanism (either in a spring force provided by the flap material or by some other spring element) that it will close itself fully as exhalation is completing. Due to the relatively high spring force provided in prior masks, the valve in prior masks generally closes while exhalation is still occurring.

However, the timing of the opening and closing of the passive valve is purely dependent upon the pressure provided by the user's breath to open the valve and keep it open. In practice, a traditional flap valve might not open and close at the ideal times in order for exhalation to exit the mask. Further, in some prior masks the opening through the valve is also too small to effectively release all the exhalation.

Powered masks work as a closed environment where loss of power would cut off the airflow and ventilation.

In some embodiments the Applicant's mask is a hybrid passive-active, or assisted passive, mask where the ease of breathing is assisted by powered operation of at least some mask components, but loss of power does not cause loss of access to airflow. In addition, with the ability to actively control mask components, airflow is actively optimized.

The Applicant proposes an innovative solution to vent the mask and relieve pressure inside the mask for improved user comfort. In some embodiments, active valve actuation is utilized to open and/or close the exhalation or outlet valve. The actuation is preferably tied to the user's breathing cycle.

With the Applicant's active valve mechanism there is more control over the timing of the change in the outlet valve position. In one embodiment the outlet valve position is controlled by an electromagnet that actively closes the outlet valve and actively holds the outlet valve closed.

At the point in the user's breathing cycle when the inhalation ends, the mask's microcontroller actuates the release of the active closure of the valve. This allows the opening of the valve in order to vent the spent or expired air from the inside of the mask during the exhalation phase.

In preferred embodiments, using an actively opened, actively closed, and actively held closed valve design, the opening and closing forces are mostly or wholly supplied by the electromagnet. This can be contrasted with prior passive masks in which a higher spring force is usually supplied by the material spring characteristics of the valve member. As the Applicant's valve, in preferred embodiments, does not rely on the spring force for opening or closing the valve, the

Applicant's valve member can be formed with a lower spring force than in prior valves, e.g. from, a thinner or more flexible material. This lower spring force means that the Applicant's valve member or flap can be opened more quickly, or in the case of loss of power, with much less pressure than in previous designs, and be closed more quickly and held closed more firmly than a non-active closing system.

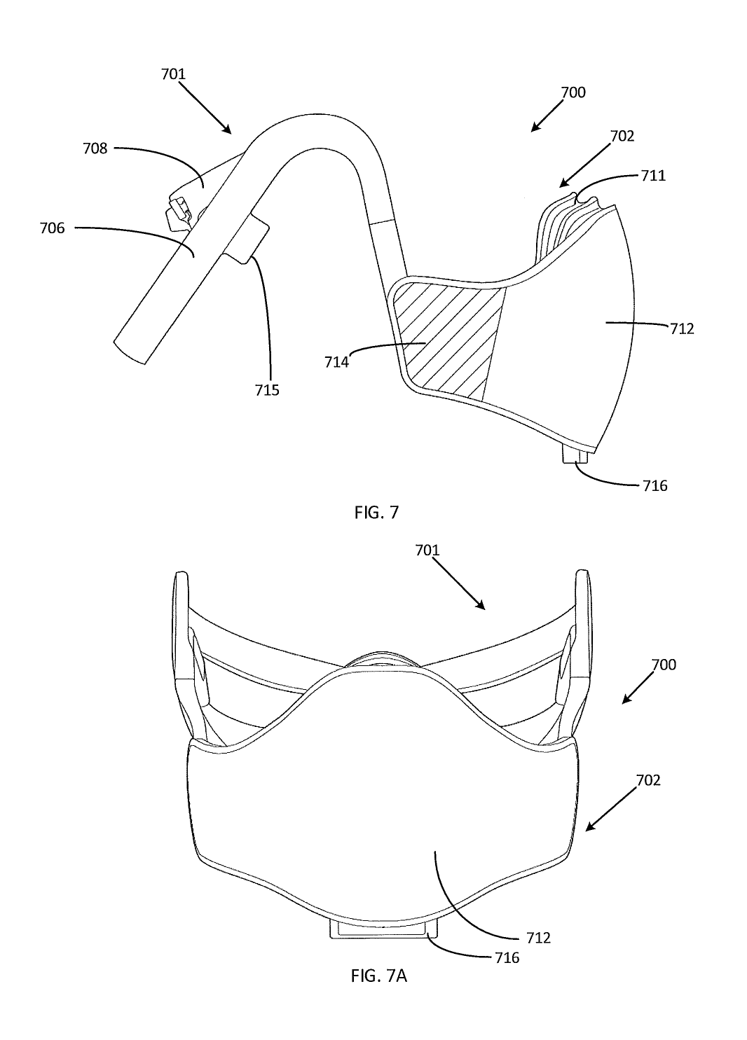

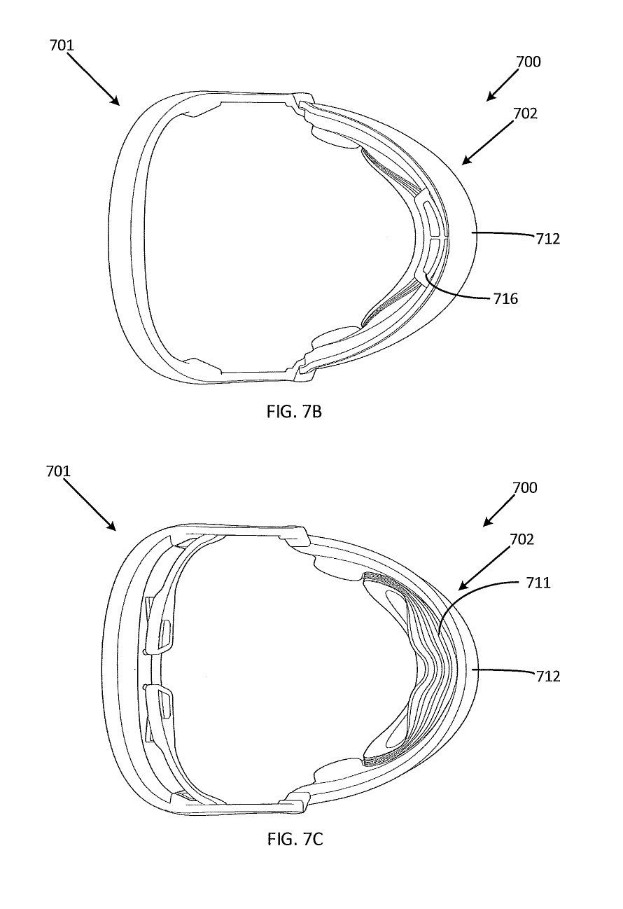

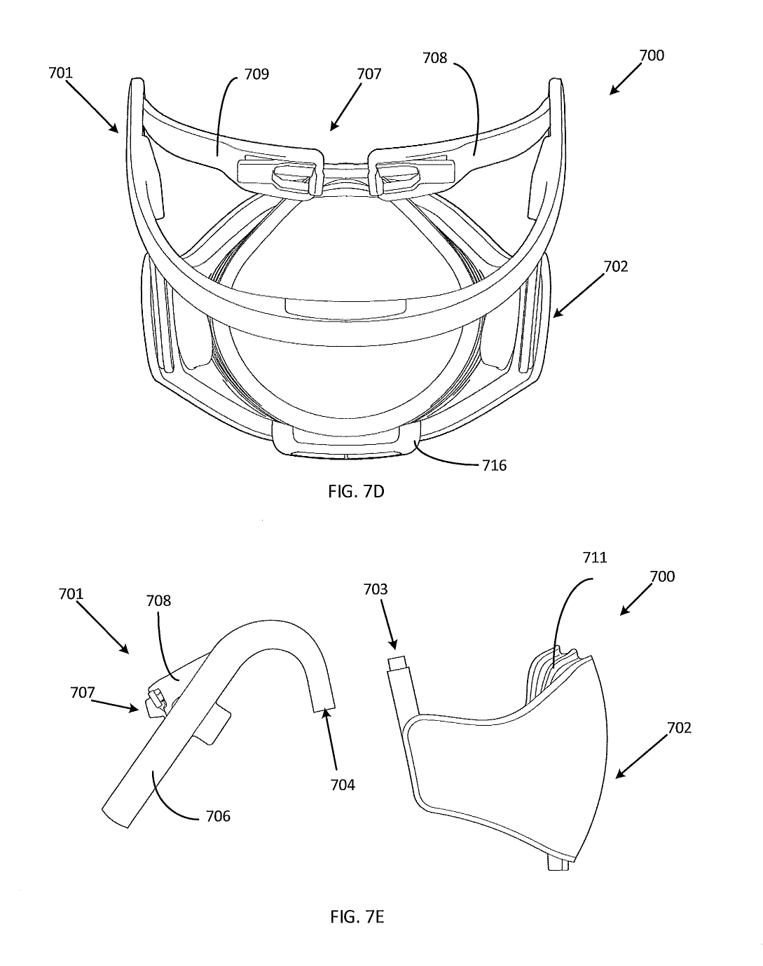

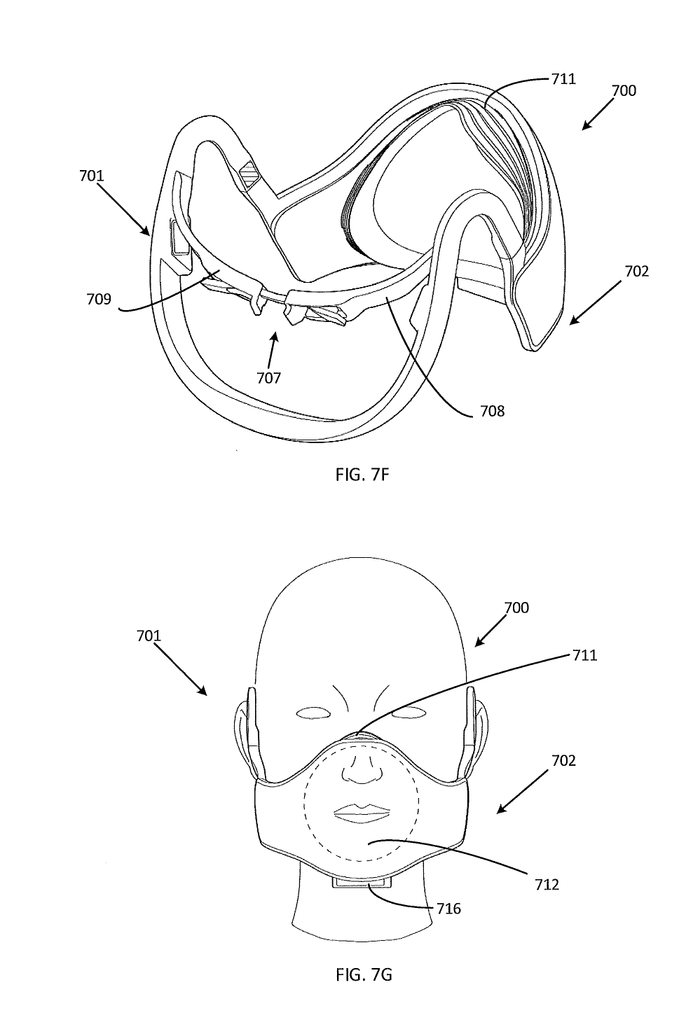

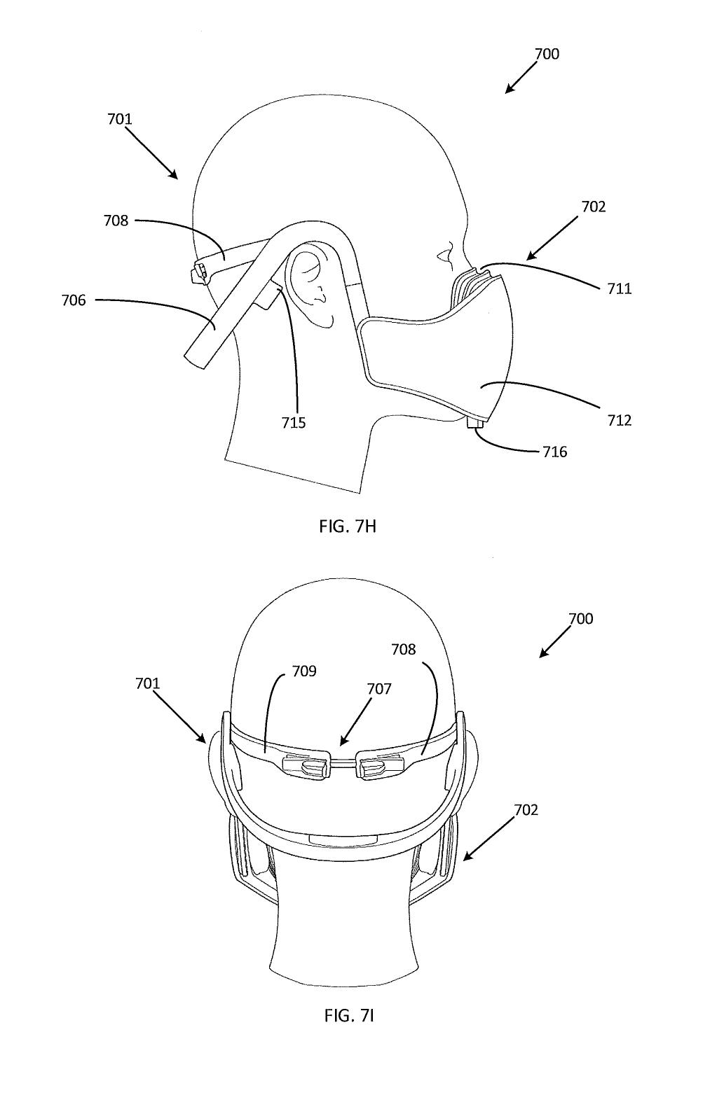

FIGS. 7 to 7J show a further embodiment of respiratory mask 700. The respiratory mask 700 includes a back frame or harness 701 and a mask portion 702.

The harness 701 and mask portion 702 may be formed in a modular fashion, with physical and electrical connections being separable as shown in FIG. 7E. The mask portion includes a male connector 703 that fits with a female connector 704 on the harness 701. The back frame 701 is therefore attached to the front frame 702 by a pair, left and right side, of magnetic secured connection sleeves 703, 704. The magnetic sleeves may have electrical contacts and an engagement feature that allow the harness or back frame and the mask portion or front frame to connect mechanically and electrically on both sides of the respiratory mask. When positioned correctly, the power, sensor and actuator connections as well as the mechanical load paths of the frame are held in place by the magnets and the sleeve fit of the male and female connectors 703, 704, together with electrical contacts or connector pins. In the preferred embodiment, there may be six connector pins per connector sleeve. The sleeves could have as few as one or as many as twelve pins per connector sleeve. When connected, there is sufficient rigidity in this connection to provide the physical connection between harness and mask portion. These electrical contacts allow transmission of power and/or data between the harness 701 and mask portion 702.

As shown in FIG. 7F, the harness 701 may include a rigid or semi-rigid curved bar 706 that extends between the female connectors 704. The bar 706 curves to fit around the back of the user's head as shown in FIGS. 7H, 7I and 7J. The bar also sits above the user's ears, which act to support the bar 706, harness 701 and the respiratory mask 700 as a whole.

The harness 701 may include an adjustment element 707 positioned on straps 708, 709 that can be adjusted to snug the back frame of the mask against the gasket element 711 of the mask portion. In other words, tightening the adjustment element 707 tends to compress the gasket element 711 against the user's face. The gasket element 711 is made from an elastomer or material that forms a comfortable seal against the facial skin of any user regardless of variations in facial shape. The gasket element 711 may have a bellows-type arrangement that accommodates a balance of forces between the adjustable snugging mechanism and the material's natural unloaded position.

The fore and aft position of the mask can be micro tuned by the pinching motion of one hand acting on the adjustment mechanism 707. The pinching motion of the adjustment increases the tension of the surrounding straps 708, 709 that pulls the back and front portions of the mask together. This increase in force compresses the front gasket against the face. The seal of the gasket 711 against the user's face can be adjusted and held in place at the correct position and level of force by user adjustment of the load using the adjustment mechanism 707.

The harness or back frame 701 may include any of the components or functionality of the corresponding elements described above with reference to FIGS. 1 to 6.

In one embodiment the front frame of the mask contains a cover element 712 that covers the internal components of the front portion of the mask. The cover element 712 is preferably easily removed and can be changed. This allows the user a choice of the same shaped element with different cosmetic treatments such as themes, characters or colors for decorative purposes. The filter retaining areas 714 of the cover element 712 have one or more apertures, preferably a pattern of multiple apertures that allow sufficient airflow through the lens material and into the air handling system of the mask. The cover element 712 may include a window or lens 713 (FIG. 7G) that is transparent in at least part of its area, to allow others to see at least the wearer's mouth. Alternatively, the cover element 712 may be transparent over its entire area. This visual connection is intended to allow viewers to see the motion of lips during speech.

In one embodiment the back frame 701 of the mask 700 may be a common size with an intended range of lateral flexure to accommodate all or several sizes of front units 702 of the mask. The front mask portion 702 may be provided in several different model configurations, such as small (child face) medium and large adult face sizes. Further, different front mask portions 701 may be provided for different classes of wearer, e.g. athletes and commuters.

In one embodiment, a bone conductive headphone may be set in the edge of the back frame 701 of the mask. In other embodiments traditional headphones, either in-ear or over-ear headphones, may be provided. In the front 702 of the mask there may be a microphone and a speaker that can be configured to allow spoken words from inside the mask to be heard outside the mask.

FIG. 7 also shows a sensor housing 715 that will be positioned over a wearer's mastoid process. A heart rate sensor and/or speaker and/or other bone conductive systems may be included in this housing 715.

The outlet valve assembly 716 is shown, and will be discussed in greater detail below.

In one embodiment, voice recognition software on the app could allow functions of the mask or a connected device, and software controlling the mask or residing on the connected device to be adjusted or controlled by voice command.

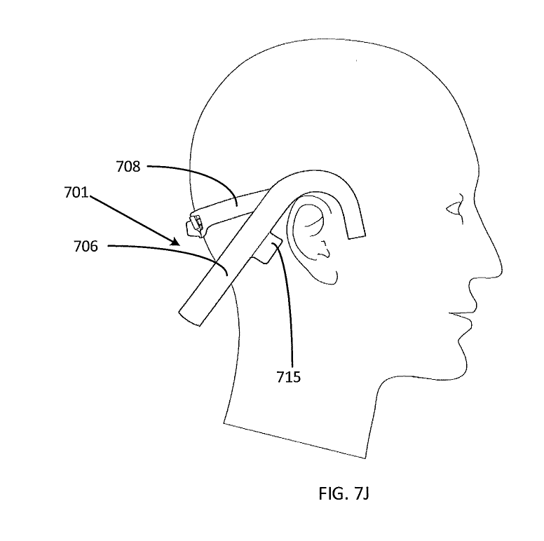

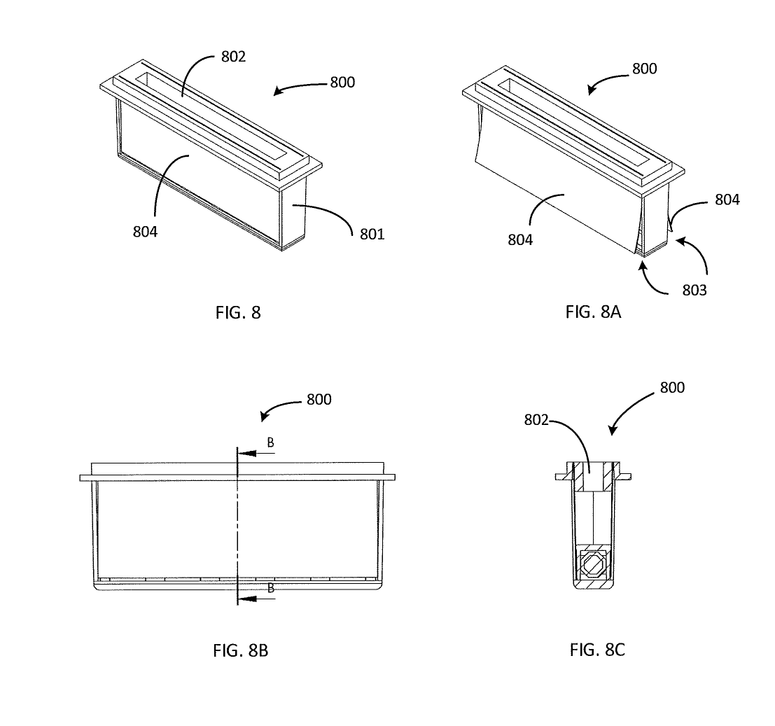

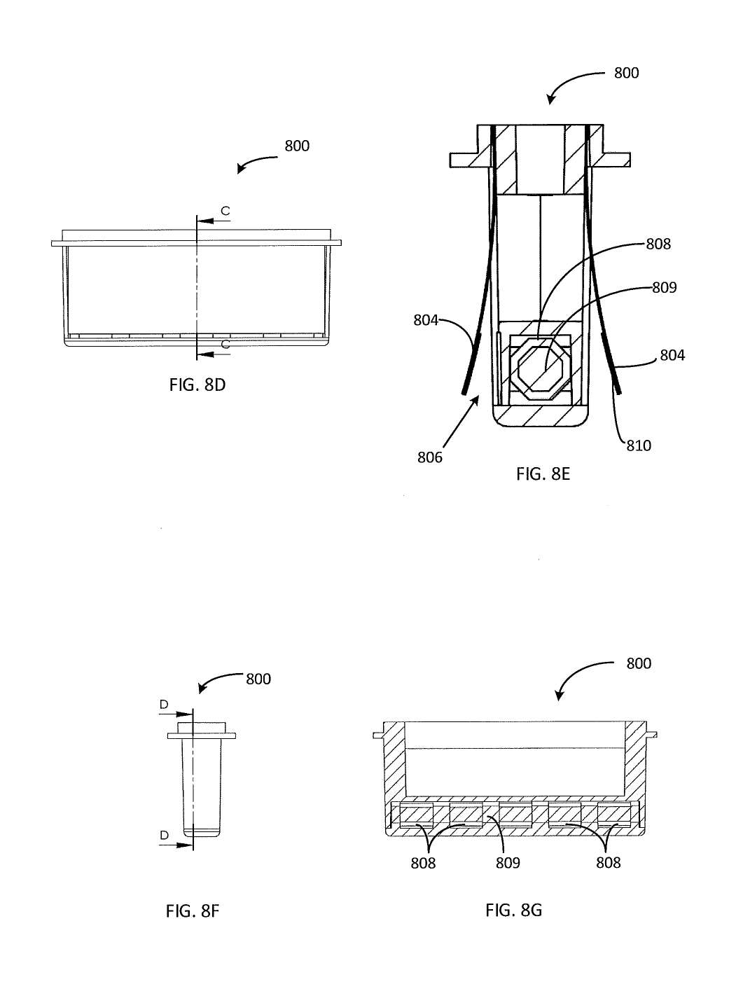

FIGS. 8 to 8G show one embodiment of outlet valve. The outlet valve 800 includes a valve body 801 with a valve inlet 802. In the assembled respiratory mask, the valve inlet 802 opens into the enclosed space within the mask. The valve 800 also includes one or more valve outlets 803. In the embodiment shown two valve outlets 803 are shown. A corresponding valve member 804 is provided to controllably open and close each valve outlet 803. Each valve member may be formed from a thin, flexible film capable of movement between the closed position of FIG. 8 (in which the valve member 804 seals against a valve seat 806 to close the valve outlet 803) and the open position of FIG. 8A (in which air can move from the valve inlet 802 to the valve outlet 803).

Movement of the valve member from the open position to the closed position may be driven by a controllable mechanism. In preferred embodiments, movement of the valve member from the open position to the closed position, and from the closed position to the open position, may be driven by a controllable mechanism.

FIG. 8G shows a number of electromagnetic coils or solenoids 808 arranged around a core 809. The solenoids 808 are electrically connected to the controller and power source (not shown in FIGS. 8 to 8G).

Each valve member 804 includes a magnetic element 810. The magnetic element may be a ferromagnetic element, such as an iron or magnetic steel element. The ferromagnetic element may be formed on or in the film of the valve member or may be attached to the valve member film in any suitable manner. In one embodiment a ferromagnetic foil may be laminated to a polymer valve member film.

When the solenoids 808 are actuated, the magnetic field produced thereby will force the ferromagnetic elements 810, and therefore the valve members 804, towards the valve seats 806 to close the valve outlets 803. This closing of the outlet valve 800 can be achieved suddenly and at a controlled specific point in time.

In preferred embodiments opening of the outlet valve may also be actively controlled by the electromagnetic mechanism. However, in alternative embodiments the outlet valve may be opened by air pressure acting from the inside of the respiratory mask onto the outlet valve. This pressure may be supplied by the user's exhaled breath and/or by pressure supplied by the inlet fan.

The use of an active closure mechanism as provided by the solenoids 808 and magnetic elements 810 allows the use of a valve member with negligible or low spring force. This means that the valve threshold is low, i.e. the force required to open the valve is low. In some embodiments the valve member may be formed from a polymer film, such as a polyester film with a thickness in the range 20 to 150 microns, preferably around 50 to 100 microns, ideally around 50 microns.

The ferromagnetic valve elements may be provided by a ferromagnetic foil, which may be laminated to the polymer film. The foil may have a thickness in the range of 0.05 mm to 0.3 mm, preferably 0.1 mm to 0.2 mm, ideally around 0.15 mm. In addition to its magnetic function, the foil acts to stiffen the valve member 804 in the region of the valve seat 803, such that a thinner material (e.g. the polymer film discussed above) can be used for the body of the valve member 804. This further reduces the spring force of the valve member 804.

The ferromagnetic core elements of the electromagnets may be formed from a rolled low-carbon steel or other high permeability material wound with suitable high conductivity wire materials such as copper. Typically, each electromagnet formed in such a way will have 1 to 500 windings of typically 0.05 mm to 0.5 mm diameter wire preferably 0.1 mm to 0.2 mm diameter wire, more preferably around 0.127 mm diameter wire.

The dimensions of the outlet valve assembly may be optimized for the particular application.

In the embodiment shown the solenoids 808 serve to actuate two valve members 804 at the same time. This provides a larger flow passage using a single electromagnetic mechanism. The larger flow passage provides less resistance to flow.

The shape of the Applicant's outlet valve with its rectangular flow passages is inherently lower-resistance than the bending-disc type found in man prior respirators, which use a disc-shaped valve member in a cylindrical passage. That prior geometry forces air to flow through one or more tight 90° turns to exit the valve.

In this embodiment the outlet valve may be forced to close by the electromagnetic arrangement of the coils 808 and magnetic elements 810. The valve may be held closed by the same mechanism. Alternatively, in some embodiments the valve may be held closed by the spring force provided by the valve member material. Further, during an inhalation phase the user's breath may act inwards to hold the valve closed.

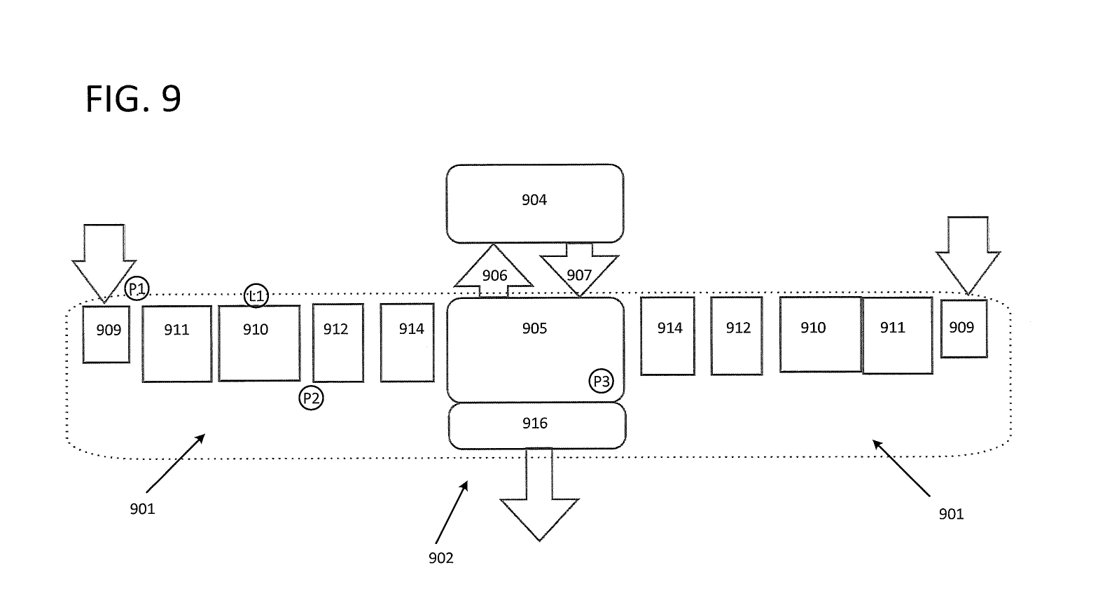

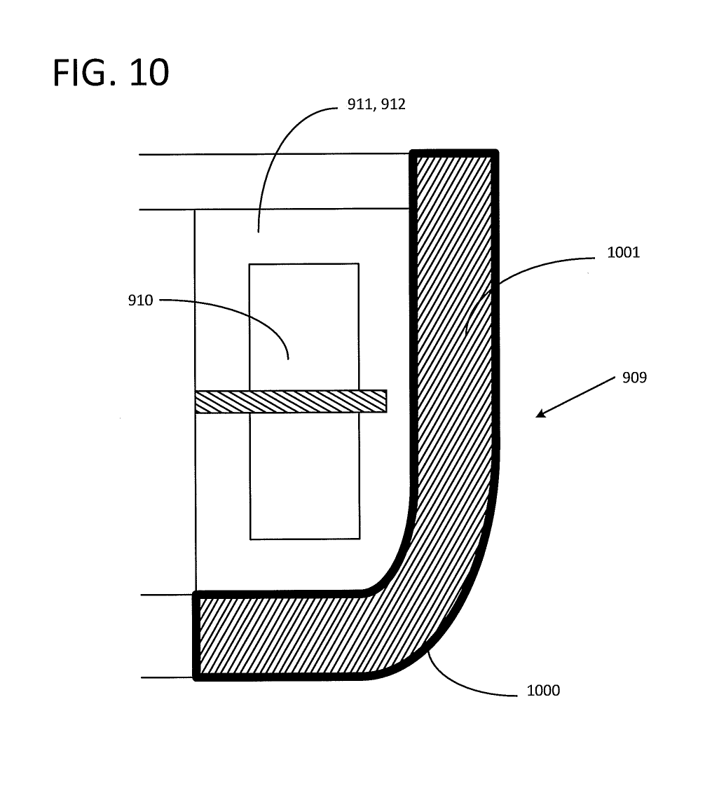

FIG. 9 is a functional diagram illustrating the working of the respiratory mask 700. In the embodiment shown the respiratory mask includes a pair of inlet paths 901 and a single outlet path 902. However, the invention is not limited in terms of the number of inlet paths and the number of outlet paths.

The position of the user's face is indicated at box 904. The respiratory mask forms an enclosed space 905 or plenum around the user's nostrils and mouth. The user inhales air from the enclosed space, as indicated by arrow 906. The user exhales air into the enclosed space 905, as indicated by arrow 907.

Each inlet path takes outside air through an inlet filter 909. Air may be drawn through the inlet filter 909 by an inlet fan 910 that is positioned within a fan box, indicated by boxes 911, 912 surrounding the fan 910. As shown, the fan 910 is preferably downstream, or to the inside of the filter 909.

Optionally, an inlet valve 914 may also be provided. This is a one-way valve allowing air to flow from the inlet path 901 into the enclosed space 905, but not allowing air flow in the other direction. This helps to protect the filters 909 from moisture in the user's exhaled breath.

An outlet valve 916 is also provided. The outlet valve 916 is controlled to allow air to flow out of the enclosed space 905, but not in the other direction.

Thus air flows into the enclosed space through the inlet paths and exits the enclosed space through the outlet path.

The inlet filter 909 is held external to the fan box 911, 912. Unlike traditional single plane filters, the Applicant's preferred filter design wraps around the outboard side of the entire fan box area and under the lower edge of the fan box in an “L” or “J” shape. The corner of the “L” or “J” may be sharp, or may be formed with a radius. This arrangement is shown schematically in FIG. 10. The inlet filter 909 consists of a filter frame 1000 holding a filter material 1001. The inlet filter slides either upwards or inwards, or a combination of the two, to attach to the outside of the fan box 911, 912, in which the fan 910 is arranged. The filter can be removed buy movement in the opposite direction. In some embodiments the filter material 1001 can be removed from the frame 1000, which has enough of a radius in in its corner that a new piece can be inserted into the frame and the frame reinserted. Alternatively, replacement filters may be supplied with the filter material already fitted to the filter frame.

This non-planar, L-shaped filter allows a greater flow area, since the filter extends around more than one side, preferably around two sides, of the fan box. Note that the cover or lens element (where used) will be positioned to the outside of the filter and will have sufficient apertures to allow flow of air. The cover or lens element is not shown in FIG. 10.

When a new filter is fitted a calibration process may or may not be performed. If calibration is to be performed, the mask may be calibrated as follows. While wearing the mask, the user is instructed to hold their breath and to issue an instruction to calibrate the new filter. This may be by selecting “CALIBRATE NEW FILTER” on the app running on the user's Smartphone. The app will instruct the mask to implement a calibration sequence in which: the outlet valve is actively opened or (in embodiments where this is applicable) released; the inlet fan is run at a predetermined power, e.g. full power, for a calibration period, e.g. for 10 seconds. Data from the pressure sensors and load information for the fan may be gathered during the calibration period, and this provides a set of reference or calibration data for the new filter. Data gathered later during normal usage, or during a filter test process, can be compared to these reference data to provide information on the current state of the filter. Further, the calibration values for substandard or counterfeit filters may fall outside of a required calibration range, allowing these filters to be identified and an appropriate warning issued during calibration.

In a further calibration method, tones may be played into the headphones to instruct the calibration sequence: Breath in and out at a specific speed or number of deep breaths in and number of deep breaths out. This sequence can calibrate the mask for that user with known filter models.

The mask may also be arranged to detect non genuine filters using appropriate codes or identifiers. For example, electrical connectors may be provided in the filter frame and the mask housing that receives the filter. Suitable microchips or other identifying elements may be provided that allow the controller to detect genuine filters.

The filter frame 1000 may be any suitable cage frame or the like. The filter frame may have sufficient rigidity to hold the filter shape when a flexible filter material 1000 is used.

In some embodiments natural wool filters may be used. The wool material provides moisture absorbent properties, helping to reduce the effects of moisture in the mask.

Further, data from the mask sensors can be used to monitor filter condition. The sensors provide information on the filter resistance to airflow. For example, sensors positioned outside and inside the filter provide a pressure difference across the filter. Together with information on the fan power or flow rate, this allows a resistance of the filter to be determined. This resistance can be monitored over time. As the filter ages the filter resistance will increase as the filter accumulates filtered particulates etc, and once it passes a threshold an alert can be issued, either within the mask or on the user's Smartphone or other device, to prompt cleaning or replacement of the filter material. Further, if the filter resistance is too small then it is likely that a filter is absent or improperly fitted, and the system can issue an appropriate alert, or lock mask functionality until a filter is properly installed.

FIG. 9 shows three pressure sensors P1, P2 and P3. Sensors P1 and P2 are positioned in the inlet path 901 and sensor P3 is positioned in the enclosed space 905. Further sensors corresponding to P1 and P2 may be provided in the second inlet path. Data may be gathered from these and any other sensors at any required rate, but preferably at around 4 to 40 Hz, preferably around 20 Hz.

In one embodiment, the mask may be started up and the controller will start the fans on full power and the outlet valve shut. The controller may read pressure sensor P1 (ambient pressure), read pressure sensor P2 (pressure in the fanbox) and pressure sensor P3 (pressure in the enclosed space or plenum) with the fan on. The pressures may be reported, for example, at 20 times per second rate. Absolute or relative pressures may be reported to the local controller or to the host running on the user's mobile device. P1 may be reported to the host for altitude and/or activity (e.g. cycling speed) measurement. P2 and P3 may be reported as relative to P1, i.e. as P2-P1, and P3-P1. If the mask is operating correctly, P2 and P3 should be negative during inhalation, and positive during exhalation. If the user is still, and has paused breathing, P1, P2 and P3 should be approximately equal. P1 may be reported as an absolute pressure value, or relative to some initial reference value of P1 (and the reference value may be periodically updated).

In preferred embodiments, the Applicant's mechanism has the ability to time the opening and closing of the outlet valve. In particular, it is possible to delay the close of the outlet valve past the end of the exhalation cycle (to allow more moisture egress) or close earlier than the purely passive system. Complete control of valve timing can be achieved through dynamic control of the timing and extent of the outlet valve opening and closing force. The Applicant's active control of the outlet valve allows optimization of the timing of the opening and closing of the outlet valve on the basis of the breathing cycle. This will allow the customization of the timing of the opening and closing of the outlet valve on the basis of the user's breathing profile.

Further, both the timing and speed of the closure can be controlled. The timing of the outlet valve opening still may start with the beginning of the exhalation phase, but the opening time may be advanced. In alternative embodiments without active opening of the outlet valve, this may be achieved by applying a pressure to the outlet valve using the inlet fan 910 or in some embodiments a further fan provided in the mask. Thus, the opening of the outlet valve can be advanced (e.g. such that the opening of the valve anticipates the beginning of the exhalation stage of the breathing cycle), or delayed slightly (e.g. by keeping the electromagnetic closing force energized, so that the electromagnet(s) apply force to the metalized edge of the flap valve member and hold it firmly shut, no matter what breathing load is applied to the valve surface, past the start of the exhalation phase. The coordination of the valve relative to the breathing cycle is such that the outlet valve timing may be moved between 0 to 25% of a total breathing cycle from the transition point from exhale to inhale or inhale to exhale. The valve can be actively opened (or in alternative embodiments released) or pulled closed. The specific moments in the cycle will be determined by the specifics of the user and their environment and activity.

The embodiment described above uses an active outlet valve with controlled opening and closing. In a further embodiment, the opening can be actuated by air pressure acting on the outlet valve, which is controllably released. The active opening mechanism may be controlled such that the outlet valve is actively opened at a desired point in the breathing cycle.

The Applicant's outlet valve does not rely solely on a spring force for closure of the valve. This allows a much lower spring force (or in some embodiments zero or even opposite spring force acting to open rather than close the valve) to be used. The significant decrease in spring force of the Applicant's valve, compared to traditional flap valves, means that, where power is lost or in alternative embodiments using air pressure combined with release of the outlet valve, less pressure from the exhaled breath is needed to fully open the valve and so less delay in the exit of the exhaled air from the mask will occur. This reduces the amount of moisture and breathed air retained in the inner space of the mask, per breath. Over many breathing cycles this results in a significantly lower amount of moisture retained in the mask.

The Applicant's valve arrangement uses less energy and parts than a fully powered valve system and has the additional safety advantage that if the power to the system fails or is interrupted, the valve system will still function in an unpowered mode. In preferred modes of operation, the active control aspects of the Applicant's mask allow optimization of flow for user needs and/or comfort, but the passive mode of operation in which no power is required remains safe.

In very general terms, the mechanics of human breathing consists of inspiration and expiration depending on the positive or negative pressure in the lungs. Inspiration/inhalation happens when the muscles around the lung (diaphragm and others) pull open the alveoli inside the lungs. The negative pressure draws air into the lungs and into the alveoli. The pressure in the lungs equalizes with the ambient pressure. The muscles then act to force air from the lungs during an expiration/exhalation stage of the breathing cycle. At the end of this exhalation stage the pressure in the lungs again equalizes with ambient pressure.

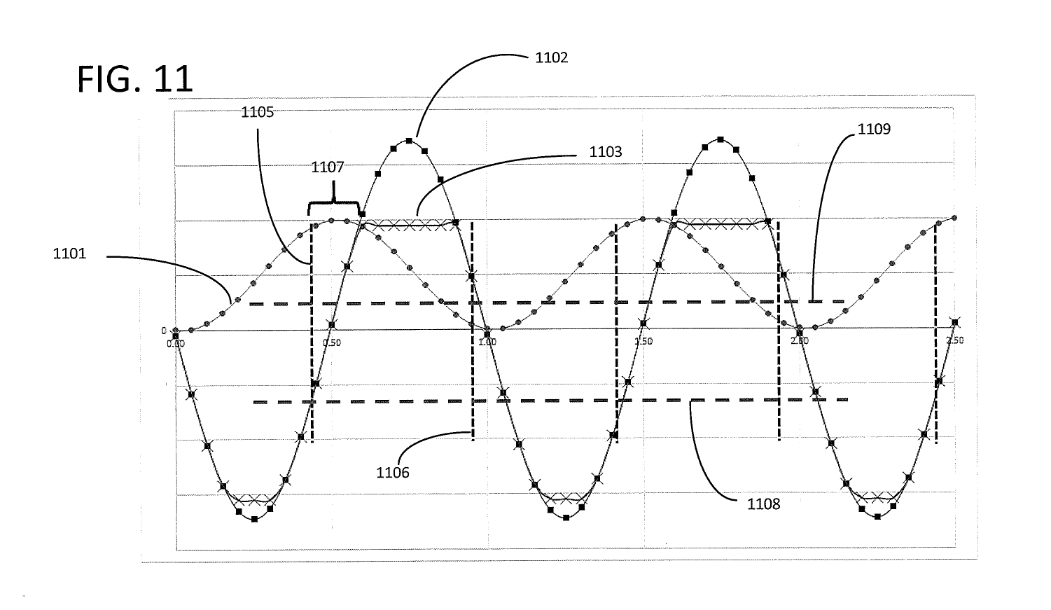

The physiology of the breathing cycle is described in the medical literature and need not be discussed in detail here. A simplified graph showing a simple breathing cycle is shown in FIG. 11. The line 1101 shows the movement of a user's diaphragm. Movement of the diaphragm away from a rest position creates a negative pressure causing inhalation of air into the user's lungs. At the end range of diaphragm movement there is a dwell time 1107, which can create a short period of minimal or zero air movement corresponding to a transition between inhaling and exhaling. The idealized movement of air is indicated by line 1102. This line includes an inhalation phase (below 0 on the vertical axis) and an exhalation phase (above 0 on the vertical axis). The movement of air is generally in time with movement of the diaphragm. A further line 1103 shows movement of air in a user wearing a respiratory mask. The peaks of the inhalation/exhalation curve are reduced due to the resistance in the mask filters, valves, flow passages etc.

In passive respiratory masks, pressure is generated inside the mask by the user's breath. Thus, in the inhalation phase a pressure inside the mask will be lower than atmospheric pressure, drawing air through the inlet filters to the interior of the mask. In the exhalation phase a pressure inside the mask will be higher than atmospheric pressure, to force air out of the mask through the outlet valve.

In the Applicant's mask one or more fans may be associated with the inlet path or paths. When the fan is running it will tend to force air through the filters into the inside of the mask. Further, in some embodiments the Applicant uses the pressure generated by the fans acting from the inside of the mask onto the outlet valve to contribute to controlled operation of the outlet valve.

In preferred embodiments the outlet valve will be actively opened before exhalation begins. In alternative embodiments in which the outlet valve is released but not actively opened, pressure may be applied by the inlet fan or fans, acting through the interior of the mask, onto the released outlet valve, causing the valve to open in advance of sufficient pressure being applied by the user's exhaled breath. (In some embodiments an outlet fan may also be provided, associated with the outlet path.)

In some embodiments the outlet valve may be actively opened or released marginally before the end of the inhalation phase, as marked by the dashed line 1105 in FIG. 11. This early outlet valve activation, ahead of exhalation, will be based on breathing rhythm comfort but should be in the range of 0.01 to 12%, preferably around 0.01 to 5%, of a typical breathing cycle. For example, if a user's breathing cycle (including inhalation and exhalation phases and dwell times) is around 5 seconds at a particular time (and this will vary with rest/exercise etc), the outlet valve may be opened around 0.5 ms to 0.6 s, preferably 0.5 ms to 0.25 s ahead of the beginning of the exhalation phase.

In preferred embodiments the outlet valve is actively closed when ambient pressure and in-mask pressure equalize at or near the end of the exhalation phase (dashed line 1106 in FIG. 11) to avoid re-entry of spent air into the mask through the outlet valve. The active closing will be timed to be at or up to 5%, preferably at or up to 2%, before the end of the exhalation cycle.

The exact timing of outlet valve opening and closure may be controlled in accordance with various breathing parameters and modes of operation, including user characteristics, user activity level, fitness, breathing rate, pulse rate etc, the specific state of the device at time of use: (e.g. filter condition, battery charge level) and environmental conditions such as weather, temperature etc.

The valve timing defined by the lines 1105 and 1106 may be set and dynamically updated based on various data. The timing may be defined based on a time relative to a point in a user's breathing cycle. The time may be absolute, or may be defined as a fraction or percentage of a breathing cycle period or phase.

The valve timing may also be defined in terms of pressure. The horizontal line 1108 represents a pressure threshold at which the controller will release or actively open the outlet valve and the line 1109 is a pressure threshold at which the outlet valve will be actively closed. Again, this pressure may be defined as an absolute value or as a fraction or percentage of some measured value, such as a peak inhalation or exhalation pressure, an average pressure in the enclosed space etc. Further, the timing may be defined based on calculations made from measured data. For example, the controller may determine a time derivative or integral of any measured value and open or release the outlet valve based on that derivative or integral. Measured data may be transformed into another domain, for example by Fourier transform into the frequency domain, and the opening or release time may be determined based on data in that domain.

In general, the controller will maintain control data in local memory. By analysis of measured information, the controller will control the active opening or release of the outlet valve based on that control data. The control data may be dynamically updated based on measured data by the controller or App on the user's mobile device. The control data may also be updated based on instructions received from the App, a server or other remote computer. These update instructions may be based on analysis of past data from that specific mask, for that specific user.

The update instructions may also be based on broader analysis of aggregated data collected from a plurality of masks worn by different users. Based on measured data and expected breathing patterns, the controller may predictively control the outlet valve.

Similar control methods may be used for control of the inlet fan or fans. Such dynamic control allows one fan to be utilized at a different rate than another if a filter or other problem produces uneven pressure loads across the inlets. In other words, flow can be preferentially directed through more functional filters.

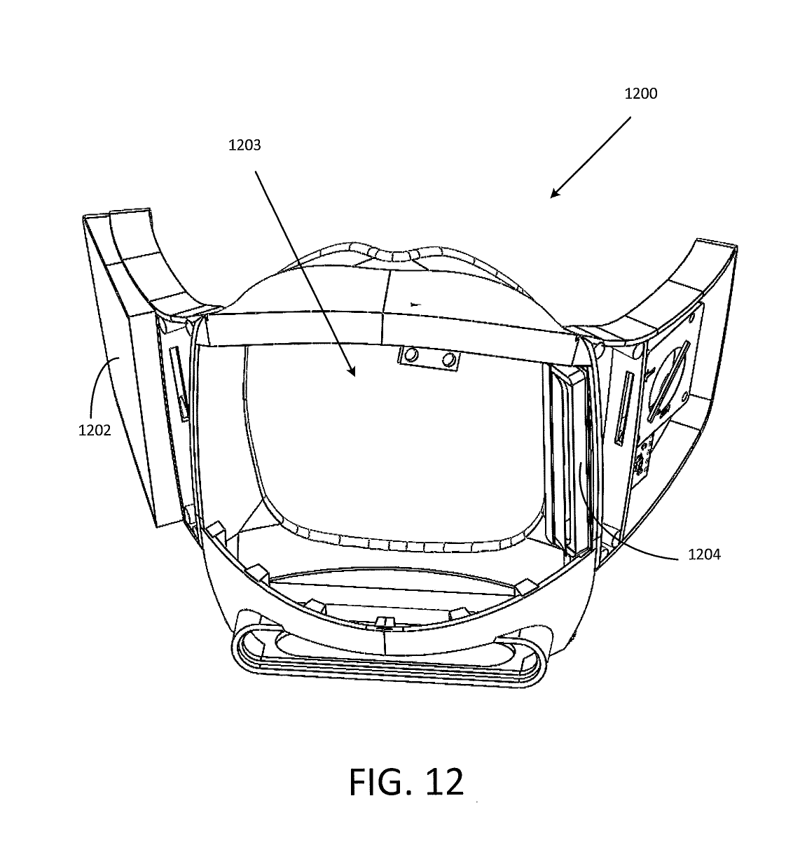

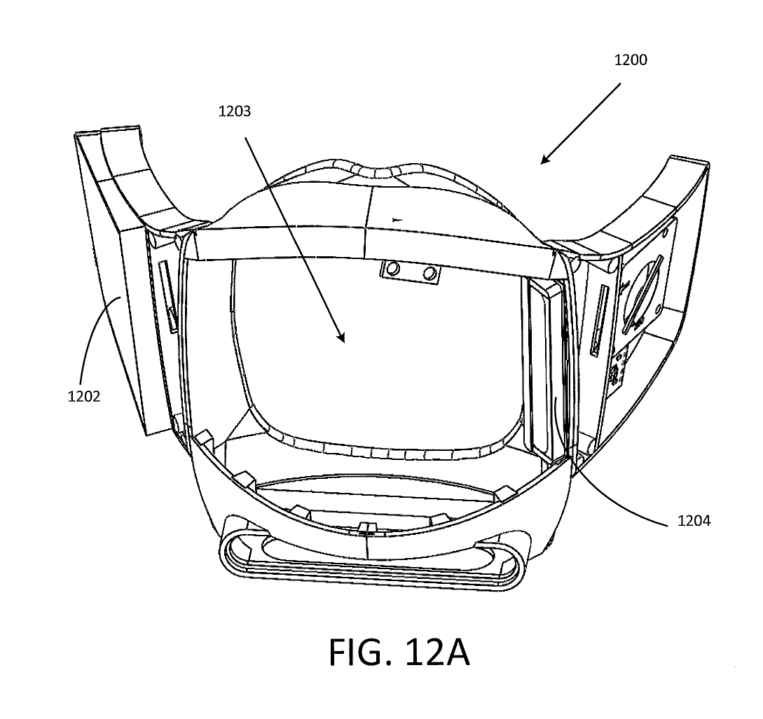

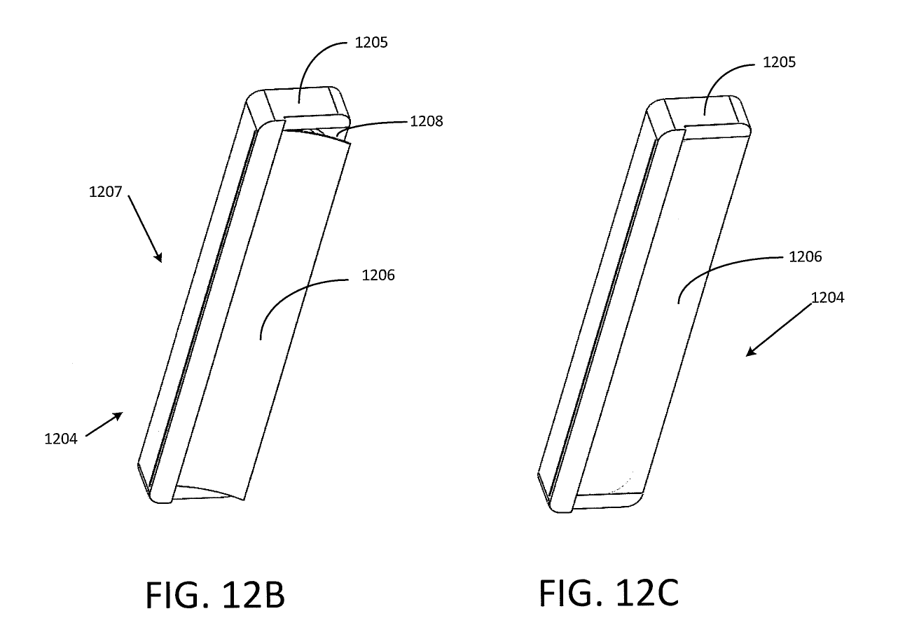

FIGS. 12 to 12C show a portion of the front mask portion 1200, with a frame defining the enclosed space 1203 and mounting points for filters 1202. On one side the filter is excluded, so that the fan 1201 can be seen. These drawings also show the position of an inlet valve 1204, which receives air from the fan 1201 and allows air flow into the enclosed space 1203 but not out of the enclosed space 1203.

The inlet valve 1204 includes a valve body 1205 with an inlet on one side 1207 (not visible in these drawings). Air flowing from the inlet tends to push the valve member or flap 1206 away from the valve seat 1208 such that the valve opens as shown in FIGS. 12 and 12B.

Air flowing in the opposite direction will tend to force the valve member 1206 against the valve seat 1208 to close the valve as shown in FIGS. 12A and 12C.

The inlet valve member 1206 may be formed from a similar thin polymer material to that used in the outlet valve, discussed above. The thin material presents minimal resistance to inwards flow. This is preferably a passive valve opening and closing under air pressure alone. However, in some embodiments actively controlled inlet valves could be used.

One embodiment will now be described, in which the outlet valve is lightly spring closed, and actively powered closed.

The control of the respiratory mask relies on the sensing of data during the user's breathing cycle. The sensed data may include the pressure at various points as the air passes through the mask. The sensed data may also include electrical parameters associated with particular mask components, for example the impedance or other electrical characteristics (e.g. power, voltage, resistance, current draw) of or across an inlet fan and/or similar or other information obtained from other moving components (e.g. fans and/or valves) to determine parameters related to the movement and/or pressure and/or pressure distribution of air in the system. This information may be indicative of the user's breathing cycle, or may be processed to provide suitable parameters associated with the user's breathing cycle. The sensed information enables the controller to control active mask elements (such as fans and/or valves) at a particular stages or points in the breathing cycle.

In one embodiment, the load on one or more fans (which may be determined by measurement of electrical impedance or another suitable electrical characteristic of the fan) allows flow characterization to be achieved by analysis of pressure differentials. Each pressure sensed by a pressure sensor is the pressure at a specific point at a specific time. The instantaneous fan load is a further piece of pressure related information that is used to provide further information relating to the flow of the air through the system. Since the pressure sensors are only passive point sensors, adding the fan as a dynamic sensor allows sensing and control to take place through the variable of the fan motion (speed and acceleration, deceleration or holding of a particular speed can all be varied and sensed). The value of the fan power input can be raised or decreased based on a predetermined pattern or information from the pressure sensors or other information such as user input, or data or instructions from external sources. The fan can be used to sense flow, but also be actively driven to change what is being sensed, unlike the purely receptive pressure sensors.

In some embodiments the outlet valve is a partially powered semi-active, controllable valve. This innovation allows active control of the outlet valve position when power is available and allows a controlled closing force to be applied to the outlet valve to close the outlet valve more quickly than in its unpowered state. Once closed, the outlet valve prevents ingress of outside air through the outlet path.

The outlet valve may be lightly biased to a closed position by a light spring force (preferably provided by the material of the valve member, although a further spring element may be used), so if no power is supplied to the valve it will be in the closed position. This light force is easily overcome by the force of exhaling breath at normal breathing levels. When unpowered, the outlet valve therefore behaves similarly to a passive valve, closing under a light bias and opening under the pressure of the user's exhaled breath. This allows the valve to operate even if there is an interruption in power, such as battery drainage or a malfunction. Unimpeded breathing will still occur. The spring force therefore assists the powered closing function when power is available and allows the unit to work functionally without power. However, in other embodiments the outlet valve may have no significant bias, or the valve may be biased towards an open position.

The powered control allows the valve to open and close at a desired time and be more rapidly and actively opened and closed than under the spring force alone. In the more active range of breathing, that is when the user is more active and their breathing cycle is faster, the timing of the opening and shutting of the vent becomes more critical to the flow management. Active closing also helps to prevent external air from re-entering the system.

Any suitable active opening and/or closing mechanism may be used, including electromechanical or electromagnetic mechanism etc. In one embodiment at least one electromagnetic coil is arranged at the edge of the valve opening. The electromagnetic coil is arranged to attract a magnetic material on the moving valve member. This magnetic material may be attached to any suitable point of the valve member, including the inner or outer or side edge of the valve member. Alternatively, the magnetic material may be introduced into the valve member in a moulding process, or the valve member may be formed of a suitable magnetic material. When the one or more electromagnetic coils are actuated, the force produced by the electromagnetic coil forces the valve towards the closed position.

Once closed, the electromagnet can actively hold the valve closed at full power or any partial amount of power down to no power since the spring force is still holding the valve lightly closed. This control allows the valve to be managed to a closed position as quickly or fully as is needed for the air management system requirements. This active control allows any breathing or atmospheric conditions that could delay or prevent the valve closing as quickly as desired to be overcome.

With this system of control, the valve can be actively closed and held at the fully closed position, preventing flow from the enclosed space out of the outlet valve, or allowed to open for venting of the breath for an appropriate portion of the exhalation cycle.

Because the spring closing force is always applied and the electromagnetic closing force can be applied to increase the force for controlled closing or stopped for spring only closing, the full range of closed position clamping forces can be achieved with this system of control. The system uses a small amount of energy and moving parts to achieve the important characteristic of having the exit valve be closed and remain closed when it is needed to oppose forces that would open it at an inappropriate part of the breathing cycle.

When opening the valve, the opening is preferably achieved by an active opening of the valve using an opposite electromagnetic force, applied to the valve member by the same electromagnets.

In one embodiment, the electromagnetic coil or coils may be energized in the reverse direction to increase the opening force on the valve. The maximum opening speed would be produced by the sum of the breathing and electromagnetic actuator force from available power minus the spring force and would be designed for a required speed and load range for users. Spring force alone is sufficient to fully close the valve, but relating the speed and position to the breathing cycles involves the powered design actively controlling the rates and timing of the valve opening and position to achieve better breathability.

In an alternative embodiment, opening may be achieved by removing the closing electromagnetic force, such that the valve is held closed only by the spring force. This allows the force of the wearer's breath to overcome the spring force to open the outlet valve. The exhaling breath of the user of the respiratory mask will apply force to the valve and overcome the light spring force.

The timing of this outlet valve active opening or release may be based on the pressure data from P2A and P3A and the fan load data F1 and in the a multi fan version the corresponding data from the other fan and sensor inputs.

Depending on the breathing inertial air mass and rhythm of the user, the start of opening of the vent may occur at the moment of exhalation commencing, or slightly before the commencement of exhalation, to avoid a back pressure lag from the valve. This value of timing is a small fraction of the total inhalation time and can be adjusted to optimize the flow characteristics of the specific user. (in the range of 0.01% to 5% of the total cycle.)

The same anticipation of the closing of the valve will have the valve close at the moment of inspiration or a small percentage of the cycle ahead of the commencement of the inspiration portion of the cycle.

In a further embodiment the outlet valve may be lightly sprung open, powered closed and also powered open. With this system of control, the valve can be actively pulled and held at the fully closed position opposing flow from the plenum chamber against internal pressure, or opened and held at any position between fully closed and fully open. Because the spring opening force is always applied and the electromagnetic opening force can be applied to oppose it for controlled closing, the full range of positions can be achieved with control.

The valve is preferably opened actively by energizing the electromagnetic coil or coils in the reverse direction. However, in some modes, to save energy, the opening may be achieved by removing closing electromagnetic force and allowing the valve to open naturally. The exhaling breath of the user of the system will add force to the opening valve to increase the opening force on the valve. The maximum opening speed would result from the spring, breathing and electromagnetic actuator forces and from available power and could be optimized by design. In some embodiments spring force alone is sufficient to fully open the valve, but relating the speed and position to the breathing cycles involves the powered design actively controlling the rates and timing of the valve opening and position.

The timing of this active opening or release may be based on the pressure data from pressure sensors P2 and P3 and the fan load data L1 and in a multi fan version the corresponding data from the other fan and sensor inputs.

Depending on the breathing inertial air mass and rhythm of the user, the start of opening of the vent may occur at the moment of exhalation commencing, or slightly before the commencement of exhalation, to avoid a back pressure lag from the valve. This value of timing is a small fraction of the total inhalation time in the range of 0.01% to 12%, preferably 0.01% to 5%, of the total cycle.

The same anticipation of the closing of the valve will have the valve close at the moment of inspiration or a small percentage of the cycle 0.01% to 12%, preferably 0.01% to 5%, ahead of the commencement of the inspiration portion of the cycle.

The Applicant's mask may have any suitable number of inlet paths, each preferably including an inlet filter, inlet fan and pressure sensors. In a preferred embodiment there are two inlet paths, one positioned on each side of the respiratory mask. The filters are preferred options but are not required for wearing or using the unit. In particular, the mask may be worn without filters while still providing entertainment and other functionality. Further, the back frame or harness portion may be worn without the front mask portion in some embodiments (FIG. 7J). However, in preferred modes of use the filters provide the desired respiratory filter function.

The maximum flow rates that are possible in the current design are achieved when the fan speed is maximized and all the obstructions to flow are minimized. So for a given model of fan, the maximum flow will be established by the maximum current that the rating of the fan unit will allow. The resistance to flow will be decreased by the use of clean filters and the best technique of breathing through the mask to maximize exhalation. When the outlet valve is open and the exhalation is as powerful as possible, then the expulsion of air flow rate will be at a maximum.

The specific maximum flow rate is dependent on filter cleanliness, breathing capacity of the user, air density and temperature, speed of travel of the user, humidity, state of precipitation and accuracy of seal fit/continuity. However, for practical applications the maximum flow rate is expected to be in the range 50 litres per minute to 400 litres per minute. The fans may produce a flow rate of 0.4 m/s each.

The electronic system included in the respiratory mask can provide various functionality to a wearing user, as well as control functions for operation of the mask. The functionality can include, for example, physiological data sensing, environmental data sensing, user input, user output, and communication network connectivity. The electronic system can be configured to communicate with an application executing on a user host device, such as a mobile phone, tablet or personal computer for transferring information gathered by the user-wearable device. The application executing on the user host device can be used to configure or update control data stored in the user-wearable device. User host devices of one or multiple users can be configured to report gathered data to a data management system, which can aggregate and store data from multiple users and perform analysis on the aggregated data.

Sensors on board the mask include pressure sensors P1, P2 and P3 and the fan load or impedance sensor L1. L1 senses load on the fan 910 (specifically on the fan motor). This data can be used to run the system hardware using a relatively simple control method. The timing and power applied to the outlet valve and inlet fans can be controlled by a microprocessor in the mask. The control parameters can be based on a predetermined set of values from known physiological data and pressure sensor and fan motor load cases. The timing of the outlet valve opening and/or closing and the timing and extent of the fan power use could be automatically adjusted based on user selection or automated selection of an operating mode, or on realtime determination of operating conditions through analysis of sensed data.

The fan draws a current that remains reasonably stable as long as the load placed on it does not change. Over a sustained period, the battery charge state will affect this, but this is understood and can be taken into account in the control arrangement. If the load on the fan motor does change by use conditions, e.g. dues to forces created by the wearer inhaling or exhaling, then the instantaneous power value of the fan will change accordingly. If the direction of the flowing breath opposes the fan (exhalation), it will decrease the motion of the fan and lower the power of the fan and the current draw, and if the direction of the flow is in the direction of flow (inhalation), it will increase the power of the fan and current draw. These differences are based on the dynamic conditions of the breathing user. Many factors will affect the extent that this effect occurs, but the range of power variation in the fan power is expected to be more than 0% and less than 20%.

If the fans are very powerful, then this effect will be more difficult to measure, since the breath output is only a proportion of the power being determined, but has a maximum value for a given individual. It is still a valid technique and allows the fan power difference to be used as an additional dynamic sensor in the system.

In general, there may be three levels of control. At a local control level, the controller within the mask may receive data from the sensors and control the fans and valves in accordance with stored control data or parameters and the sensed data. Control buttons may be provided on the mask to allow user input to the on-board controller. At a secondary control level, the app may be used to control mask functions over a wireless link. User input may be received by the app. The app may issue control instructions and/or instructions to alter or update control data or parameters to the on-board controller. At a third control level, the remote computer may issue instructions to the app or directly to the mask. These may be control instructions and/or instructions to alter or update control data or parameters. When the app receives instructions from the remote computer it will issue its own instructions to the on-board controller.

For example, in one embodiment the app could allow a user to select a maximum throughput mode or a maximum battery life mode.

In maximum throughput mode, the fans could be run at full power for inhale and exhale, providing maximum airflow in a simplified sport mode and the vent could be closed by pressure sensing with no timing adjustment based on a single pressure threshold variable change. In other words, the outlet valve could be actively closed when exhalation pressure sensed by sensor P3 drops below a threshold.

In maximum battery life mode, the fans may be used at some highly reduced percentage of full power for inhalation only and the fan power is switched off for the exhalation phase. The valve may be actively shut at the end of the exhalation phase based on a single pressure sensor reading. This arrangement may be the minimum system use of battery that is possible. However, a full passive mode may also be provided where there is no powered use of either fans or outlet valve, with the mask operating as a passive system. In this mode the user draws air through the inlet filter and forces air through the outlet valve by their breathing pressure.

In one embodiment the level of power consumption or contribution to breathing could be adjusted by user input to a desired level, e.g. a percentage value, or on a sliding scale. This could be done by input on control buttons on the respiratory mask or using the app. In such modes, there may be no sensing of the individual user's biometrics, but the user could accept the system minimum flow as the most efficient option for power use, or increase fan volume manually to add to the flow (but decrease battery life).

In a further mode of operation, the user may be allowed to alter the range or timing of the strategy for that user. For example, the user could adjust for maximum comfort, where the fans might be forced to remain running at higher level of throughput than the battery saving settings recommend, or choose maximum battery life mode where the active assisting of the breathing is less active, but the system will run for longer. The assisted passive strategy can be adjusted for maximum air throughput, maximum battery life, or a range of values between the two extremes. The app may provide sliders for the conditions of battery life and air volume, the user can set the usage factors that they want.

The modes of operation can also take into account other inputs such as the basic App data, and data supplied by the user, (e.g. height, weight, age etc). This is one level of customized control above the mask only operation.

Further activity modes may be provided, such as “Athlete”, “Sport”, “Commuter” modes, in order to optimize air flow and power usage for a particular activity.

A blended control mode can allow the user to set one or more factors in the App and then then a Web-based personal data analyzer will blend these factors with known system parameters and optimize the control arrangement for the specific user.

As an example, if the user has trained for three weeks and their workouts are typically 40 minutes long, the data of the previous uses can be used to suggest a power and breathability outline to the user before the workout to fit fan speed and timing and valve timing to the expected work out. If the system user agrees to the plan, the energy and timing strategy can be optimized for the expected length of workout. This analysis may be done either in the App or at a remote computer. The maximum breathing rate of the system will be known for that user and much of the calculation will not need to be repeated for controlling the unit. All of this will reduce battery use, so the fan power can be optimized for the intended workout length.

All the sensors in the system may produce signals when they are powered up. It is possible that all the data of the outputs of the sensors will be collected and stored for subsequent processing. Meaning can be inferred by processing and analyzing multiple data points to determine a piece of coherent information. The use of context and relating the data points to one another assigns meaning to individual data values. e.g. a single heart rate value at a moment does not tell you if the user's heart rate is going up, down or staying steady.

Some data can be at least partly processed before it is stored. For example, sensor data from the heart rate sensor may be processed on-board the respiratory mask to provide heart rate values. Data may also be assembling it into relevant packet sizes to suit a secondary process. The mask may have limited storage. Some data may be more efficiently processed off the mask, where greater processing power and memory is available. Further, it may be desirable to retain raw data from some sensors.

With this concept in mind, sensor data may reside: in the mask—where there is a limited on-board memory such as flash drive; on the user's Smartphone or other mobile device or computer; on a remote computer (e.g. on the Cloud). Some data may be pushed the App and stored as raw or processed data on the hosting phone. Similarly, some raw or processed data may be pushed from the mask to the phone app to the Cloud.

Once data is transferred to the phone app, the significantly greater processor power and memory of the phone allows processing and storage of data from many events and activities. In a preferred embodiment of the current invention, the phone will be linked by blue tooth wireless connection to pass data to the app for storage and processing. Some of the data could be processed “on the fly” in real time, and other aspects of the data might be processed at a slower rate than real time or stored and processed as a secondary operation.

Further data analysis of a specific mask user could be achieved by performing analysis on that mask data or combining the individual mask data with other mask users or other health and data to send back modified data or operating instructions to the mask. Changes in settings or performance of the mask could be generated by analysis requiring more computing power than available in the mask or phone. Changes can be based on data collected from an individual mask and relating to an individual user, and/or changes may be based on data aggregated from a plurality of masks.

Over an extended period, the cloud data base of users could lead to refinements in the mask hardware and hardware operating systems, and in particular in the control parameters or control data stored on-board the mask.

In some embodiments, data will be gathered by the mask and be passed from mask to app, from app to cloud, from cloud to app, and on a limited basis from app to mask.

In general, the complexity and volume of data held in the mask will be less than for data held in the app, which will be less than for data held in the cloud.

Three modes of operation will now be described. The control system may implement any number of modes of operation as required.

“Athlete” mode—for athletes, top priorities for the respiratory mask include: maximizing air flow (so the fan RPM will be high, or at maximum for much of the time); maximizing data capture (so high or maximum sensor capture rate). A higher capacity battery may be provided for use by athletes. Entertainment features may be excluded or disabled. Additional on board memory may be provided.

In athlete mode the fan will need as much of the battery capacity as it can have, so the controller will need to optimize the use of power. During exercise the athlete's breathing cycle is likely to increase in breathing volume and rate from rest values to peak values. The fan power will be higher for an athlete breathing deeply than for an athlete at rest.

During the early stages of an exercise session, the fan will be on full when the athlete is inhaling and may drop to ¾or ½of full power while the athlete is exhaling. As the stress of the workout creates deeper breathing, the fans may go to full power through the full breathing cycle.

The outlet valve may be powered to full close during at least most of the inhalation cycle (see FIG. 11). The valve is opened at the point shown by the vertical dotted line 1105. This point may precede the beginning of the exhalation phase, and in some embodiments may fall a very short time before the end of the inhalation phase. The valve may be actively opened prior to the start of exhalation or may be pushed open when pressure from the user's exhalation begins to reach the valve. Further, pressure acting from the inlet fan through the enclosed space will also act against the outlet valve and may in some embodiments cause it to open in advance of exhalation pressure. In still further embodiments the inlet fan pressure acts to assist the exhalation pressure in opening the outlet valve.

The outlet valve is then re-powered to close at the vertical dotted line 1106. The valve closure force may then be held at full power for at least most of the inhalation cycle. The exact point of the valve closure may depend on the athlete and may be adjusted by bio feedback from the app and the cloud. There is opportunity here for the analysis to confirm the best strategy for a given user by running “partial timing experiments”. The timing of the valve closing and opening can be noted relative to the fan load and/or pressure sensor data. By varying the time, e.g. by 1 ms in each direction from the starting point, and noting the load of the fan and/or pressure sensor output, a curve of timing to load could be plotted to see if there is an optimal time to open and close the valve in terms of reducing the fan load or allowing more fan efficiency.

“Simple commuter” mode—the system may priorities efficiency to maintain battery life. User comfort is key. Entertainment systems (e.g. sound system, headphones etc), and mobile phone functionality etc are enabled. The system may be arranged to provide notifications relating to travel options, train delays etc.

The commuter is very unlikely to be breathing at elevated levels. This relatively shallow breathing depth will draw less current from the fans. and the volume of air passing through the system will also require less fan effort and less valve effort.

The fan may operate at around ⅓to ½of the maximum rate during the inhalation phase. The fan may be off during the exhalation phase, although it is preferably still free to spin in the air flow through the inlet path. The valve will actuate at the same timing as the other modes, but the force needed for certainty of closure will be smaller than with the higher rate of breathing expected in Athlete mode, for example. The valve closure mechanism may therefore operate at lower power than in Athlete mode.

Generally, it is desirable to maintain a high data acquisition rate and density. However, in some modes (e.g. commuter mode) or in order to save power the data acquisition rate and density may be reduced.

“Sports” mode—this may be a mode intermediate between the commuter and athlete modes, suitable for amateur sportspeople, for example. The Sport mode may be suitable for a user who exercises but is not pushing the boundaries of high volume or exertion.

The fan may operate at around ½to ¾of maximum power during the inhalation phase and at around 0 to ⅓of maximum power during the exhalation phase. Data may be gathered at a higher rate than for commuter mode. All data may be sent to the app with storage for later connection to cloud.

In preferred embodiments the Applicant's outlet valve is controlled to open just before the commencement of expiration. The outlet valve may be controlled to open just before inspiration /inhalation. The Applicant's mask may use dynamic control of the rate or power of the fan motors and this may not be on or off only. Preferred embodiments use both controlled timing of the outlet valve actuation (closing and release or active opening) and dynamic control of fan power.

The data gathered by the mask can be used to alert wearers to the level of pollution that they are experiencing over prolonged periods based on rates of filter clogging or changes in sensed pressures/fan loads. Pressure differentials may be monitored for this purpose at a given frequency. Filter data collection can be tied to the location of the mask and the date of exposure through the app/web analysis. By logging location of the phone or mask and tying the pollution rate data in the mask to the location of occurrence, the system could provide users with recommended locations to avoid, or filter life expectancies, or recommended filter change schedules.

In fitness modes, the Applicant's system may provide fitness goal or physiotherapy audio features instructing a workout routine. Such features may combine web analysis, app data collection and porting, sound features and programming.

If the user has a target heart rate, the mask/app/or web control systems can introduce audio instructions, such as a breathing metronomic function to guide the user to a suggested breathing rate in order to achieve a desired heart rate. The tones signal the points where the user should breath in and out to adjust their heart rate to a set level. Similarly, audio instructions can assist the user to hold the breathing rate constant while the system monitors the change in heart rate over time. Both these modes allow the mask to help train the user to target their heart rate by breathing training and response. The breathing cues can be words or tones or clicks or haptic cues.

In one embodiment the respiratory mask may issue alerts based on the sensor outputs. For example, if heart rate, temperature or pressure sensors stray from a normal range, an alert may be sent to ask the user to confirm their health. If there is no response, a further alert may be sent to emergency services, including position information for the user.

Once the rhythm of the user's breathing is identified, fan levels could be adjusted to minimize the fan use in favor of prolonging the battery life. For any given pattern of breathing and fitness, power use of the fans and valves can be optimized for the maximum comfort or length of battery life, or any desired weighting between comfort and battery life.

In some embodiments the cleanliness of filters may be monitored by logging and tracking the flow resistance by pressure and/or differential monitoring (such as fan impedance/speed). The App may ask a user to perform a calibration sequence when filters are new. The pressure differential between P1, P2 and P3 as well as the fan resistance compared to P2 can be used to establish a recorded “clean state” for the owner/user of the mask. This recorded data will be used as the benchmark to monitor filter quality and longevity. Upon each use of the mask, the electronics of the mask will log the time of use. In the preferred embodiment the number of breaths and some reference to the interpolated volume will be logged. In addition to this data, in the preferred embodiment, the location of the user could be retained to relate position to environmental conditions. When the sensor data from P1, P2, and P3 indicates that the filter is not performing correctly, the mask or App will take the appropriate action that the mask or App has been programmed to undertake, such as notifying the user that the filters need changing, or simply notifying the user of the filter state. Repeated reminders may be issued if the user does not change the filter.

For health and safety of the users, in some embodiments the identity and “breathing behavior” of the user may be established at the start of use. Without a thorough cleaning, it is not advisable that the mask be shared among multiple users, so during initiation of the unit, in the preferred embodiment, a calibration sequence may take place during approximately 10, 30 and 60 second sampling intervals. The breathing pressure values and timing values may be collected and stored as a reference user profile. If a future user does not match the owner breathing profile, an alert may be sent to the registered owner of the mask that a suspected non owner is using the mask. An alert in the mask electronics could also be generated to tell the user that the mask is not theirs (two masks could look the same).

The invention relates mainly to self-contained respiratory masks that are worn over a user's face. All components of the mask are worn on the user's head, with no external hoses or the like required for connection to external filters or fans. However, as is clear from the above description, wireless communications connections may be provided from the self-contained respiratory mask to external devices, Smartphones, computers, communications networks etc.

While the present invention has been illustrated by the description of the embodiments thereof, and while the embodiments have been described in detail, it is not the intention of the Applicant to restrict or in any way limit the scope of the appended claims to such detail. Further, the above embodiments may be implemented individually, or may be combined where compatible. Additional advantages and modifications, including combinations of the above embodiments, will readily appear to those skilled in the art. Therefore, the invention in its broader aspects is not limited to the specific details, representative apparatus and methods, and illustrative examples shown and described. Accordingly, departures may be made from such details without departure from the spirit or scope of the Applicant's general inventive concept.

1. A self-contained respiratory mask including:
i. a mask body configured to be positioned over at least part of a user's face, the mask body, in use, cooperating with the user's face to define an enclosed space covering at least the user's nostrils and mouth;
ii. at least one inlet path for entry of air into the enclosed space, the inlet path including an air inlet formed in the mask body and an inlet filter;
iii. at least one outlet path for exit of air from the enclosed space, the outlet path including an outlet formed in the mask body and a controllable outlet valve;
iv. a power source;
v. one or more sensors configured to sense one or more parameters indicative of a breathing cycle of the user; and
vi. a controller configured to control the outlet valve in accordance with the sensed parameters.

2. A self-contained respiratory mask as claimed in claim 1 wherein each outlet valve includes a valve member having one or more magnetic elements, a valve seat, and an electromagnet configured, when actuated, to create a force acting on the magnetic elements to drive movement of the valve member relative to the valve seat.

3. (canceled)

4. (canceled)

5. (canceled)

6. (canceled)

7. (canceled)

8. (canceled)

9. A self-contained respiratory mask as claimed in claim 1 wherein the controller is arranged to control the outlet valve, to close and hold closed the outlet valve.

10. (canceled)

11. (canceled)

12. A self-contained respiratory mask as claimed in claim 1 wherein the controller is arranged to control the outlet valve, to actively open the outlet valve.

13. (canceled)

14. A self-contained respiratory mask as claimed in claim 1 wherein the controller is configured to control timed closure of the outlet valve.

15. A self-contained respiratory mask as claimed in claim 1 wherein the controller is configured to control timed release or active opening of the outlet valve.

16. A self-contained respiratory mask as claimed in claim 1 wherein the inlet path further includes an inlet fan and wherein the controller is also configured to control the inlet fan in accordance with the sensed parameters.

17. A self-contained respiratory mask as claimed in claim 16 wherein the controller is configured to control the inlet fan such that sufficient pressure is generated to cause airflow into the enclosed space such that pressure acting outwards from the enclosed space causes the outlet valve to open or to remain open.

18. A self-contained respiratory mask as claimed in claim 16 wherein the controller is configured to control one or more of: a power level of the inlet fan, and a power level applied to the closure of the outlet valve.

19. A self-contained respiratory mask as claimed in claim 16, wherein the controller is configured to control flow parameters of the inlet fan over the user's breathing cycle.

20. A self-contained respiratory mask as claimed in claim 1 wherein the controller is configured to control the outlet valve to: close the outlet valve at a desired point of the breathing cycle; and release and/or open the outlet valve at a further desired point of the breathing cycle.

21. A self-contained respiratory mask as claimed in claim 20 wherein the controller is configured to dynamically update the desired point and/or the further desired point of the breathing cycle.

22. A self-contained respiratory mask as claimed in claim 1, wherein the controller is configured to control the outlet valve and/or the inlet fan to open the outlet valve at or before the beginning of the exhalation phase of the user's breathing cycle.

23. A self-contained respiratory mask as claimed in claim 22, wherein the controller is configured to control the outlet valve and/or the inlet fan to open the outlet valve 0.01% to 12% of a breathing cycle before the beginning of the exhalation phase of the user's breathing cycle.

24. A self-contained respiratory mask as claimed in claim 22, wherein the controller is configured to control the outlet valve and/or the inlet fan to open the outlet valve 0.01% to 5% of a breathing cycle before the beginning of the exhalation phase of the user's breathing cycle.

25. A self-contained respiratory mask as claimed in claim 1, wherein the controller is configured to control the outlet valve and/or the inlet fan such that the outlet valve remains open past the end of the exhalation phase of the user's breathing cycle.

26. A self-contained respiratory mask as claimed in claim 1, wherein the controller is configured to control the outlet valve such that the outlet valve is closed before the beginning of the inhalation phase of the user's breathing cycle.

27. A self-contained respiratory mask as claimed in claim 1 wherein the inlet path further includes a one-way inlet valve positioned downstream of the inlet filter, the inlet valve being configured to allow flow through the inlet path into the enclosed space but not out of the enclosed space.

28. (canceled)

29. A self-contained respiratory mask as claimed in claim 1, including a communications interface; wherein the controller is configured to:
i. receive sensed parameters from the one or more sensors; and
ii. communicate the received parameters to an external device.

30. A self-contained respiratory mask as claimed in claim 1, wherein the controller is configured to:
i. maintain local control data in the memory;
ii. update the local control data;
iii. receive sensed parameters from the one or more sensors; and
iv. control the controllable inlet blower and/or the controllable outlet valve in accordance with the updated local control data and sensed parameters received from the one or more sensors.

31. A self-contained respiratory mask as claimed in claim 30, further including a communications interface, wherein the controller is further configured to:
communicate usage data via the communications interface to an external device;
receive, from the external device, update instructions based on the communicated usage data; and update the local control data in accordance with the update instructions.

32. (canceled)

33. (canceled)

34. (canceled)

35. (canceled)

36. (canceled)

37. (canceled)

38. (canceled)

39. (canceled)

40. A self-contained respiratory mask including:
i. a mask body configured to be positioned over at least part of a user's face, the mask body, in use, cooperating with the user's face to define an enclosed space covering at least the user's nostrils and mouth;
ii. at least one inlet path for entry of air into the enclosed space, the inlet path including an air inlet formed in the mask body, an inlet blower and an inlet filter;
iii. at least one outlet path for exit of air from the enclosed space, the outlet path including an outlet formed in the mask body and an outlet valve;
iv. a power source;
v. one or more sensors configured to sense one or more parameters indicative of a breathing cycle of the user; and
vi. a controller configured to control the inlet fan in accordance with the sensed parameters.

41.-56. (canceled)

57. A self-contained respiratory mask including:
i. a mask body configured to be positioned over at least part of a user's face, the mask body, in use, cooperating with the user's face to define an enclosed space covering at least the user's nostrils and mouth;
ii. at least one inlet path for entry of air into the enclosed space, the inlet path including an air inlet formed in the mask body and an inlet filter;
iii. at least one outlet path for exit of air from the enclosed space, the outlet path including an outlet formed in the mask body and an outlet valve;
iv. a power source;
v. one or more sensors configured to sense one or more parameters associated with a wearer's physiology and/or breathing cycle;
vi. a communications interface; and
vii. a controller configured to:
a. receive sensed parameters from the one or more sensors; and
b. communicate the received parameters and/or processed data based on the received parameters to an external device.

58. (canceled)

59. (canceled)

60. (canceled)

61. (canceled)

62. A self-contained respiratory mask as claimed in claim 1 including a front mask portion and a back harness portion separable at a number of connectors, at least one of the connectors providing separable mechanical and electrical connection between the front mask portion and the back harness portion.

63. A self-contained respiratory mask as claimed in claim 1, configured to operate in a purely passive mode in the event of power failure.

64. (canceled)

65. A self-contained respiratory mask as claimed in claim 1, wherein the controller is arranged to implement one of a plurality of active control modes including at least a high power mode in which mask function is prioritized and a low power mode in which power source life is prioritized.

66. A self-contained respiratory mask as claimed in claim 65 wherein the controller is configured to change the control mode based on input from the user.

67. A self-contained respiratory mask as claimed in claim 65 wherein the controller is configured to change the control mode based on data received from the one or more sensors and/or information on remaining power source charge.

68. A self-contained respiratory mask as claim 1 wherein the one or more sensors include one or more pressure sensors.

69. A self-contained respiratory mask as claimed in claim 68 wherein the pressure sensors include a first sensor positioned to the outside of the inlet filter and inlet fan and a second sensor positioned to the inside of the inlet filter and inlet fan.

70. A self-contained respiratory mask as claimed in claim 68 wherein the pressure sensors include a pressure sensor in the enclosed space.

71. A self-contained respiratory mask as claimed in claim 1 wherein the one or more sensors include an electrical sensor configured to sense one or more electrical characteristics of the inlet fan.

72. (canceled)

73. (canceled)

74. (canceled)

75. (canceled)

76. A self-contained respiratory mask as claimed in claim 1 further including a communications interface for communications with a user's mobile device, Smartphone and/or computer.

77. (canceled)

78. A self-contained respiratory mask as claimed in claim 77, wherein the physiological sensors comprise one or more of: temperature sensors; a body temperature sensor; an air temperature sensor positioned to sense temperature of exhaled air; an air pressure sensor.

79. A self-contained respiratory mask as claimed in claim 1 including a GPS receiver module.

80. A self-contained respiratory mask as claimed in claim 1 including memory configured to store data gathered by at least one of the sensors.

81. (canceled)

82. (canceled)

83. (canceled)

84. (canceled)

85. (canceled)

86. The user-wearable device of claim 1, wherein one or more sensors include one or more physiological sensors, wherein at least one of the physiological sensors is positioned within the respiratory mask portion.

87.-97. (canceled)
